SEQUENCE LISTING (49 sequences)

SEQ ID NO: 1 - DNA, 215 bp, Rattus norvegicus
  700251567H1
cgaccattccctatacactggcagtctctggtacacaaccatccggcgggagtggtattatgangtgatcattgtacgtgtagaaatcaatggncaagatcngacnatggactgcacagagtacaactatgncaagagcatcntggangtggaccaccaacttcgtttgcccaagaaagtatttgnagccnnnnnatccttcagnagcctcctng

SEQ ID NO: 2 - DNA, 423 bp, Rattus norvegicus
  701432228T1
taaataacttcctgtcaaggattatagattttgatacaaataatctgatgtgtttcttctgaacacatttaagaaactaaacattaccatgcatggggcatgatggaagattagagagaatcttgacaccccaagaaatggctgatcttatcatttggttttgggtaacctgctctcctgtgctaggacatagtgtctgtctctttttcttttagatcattggtggtatcgaccattccctatacactggcagtctctggtacacacccatccggcgggagtggtattatgaagtgatcattgtacgtgtagaaatcaatggtcaagatctgaaaatgggactgcnaagnaggtaataaactggaaggggacccagtgagagtaagaggttaaatcccantgtgngagaaccatgagggttgg

SEQ ID NO: 3 - DNA, 275 bp, Rattus norvegicus
  700769871H1
gttctgtgatggctctgcaggccctgcccttcaccttagggagtgctgtgtgattgccaactttctgccttggcacagccctaacctcagcccaggtcctgtgcctccaccctctcctccaccctcatggttctctcacactgggattaacctcttactctcactgggtccccttccagtttattacctccttgcagtccattttcagatcttgaccattgatttctacacgtacaatgatcacttcataataccactcccgccggatgggtgtg

SEQ ID NO: 4 - DNA, 271 bp, Rattus norvegicus
  700769257H1
gttctgtgatggctctgcaggccctgcccttcaccttagggagtgctgtgtgattgccaactttctgccttggcacagccctaacctcagcccaggtcctgtgcctccaccctctcctccaccctcatggttctctcacactgggattaacctcttactctcactgggtccccttccagtttattacctccttgcagtccnttttcagntcttgaccattgatttctacacgtacaatgatcacttcatantaccactcccgccggatggg

SEQ ID NO: 5 - DNA, 276 bp, Rattus norvegicus
  701188357H1
gaactcttctgtgttttcattactgtacttaagctctatgctcaggttctgtgatggctctgcaggccctgcccttcaccttagggagtgctgtgtgattgccaagtttctgccttggcacagccctaacctcagcccaggtcctgtgcctccaccctctcctccaccctcatggttctctcacactgggattaacctcttactctcactgggtccccttccagtttattacctctttgcagtccattttcagatcttgaccattgattnctacac

SEQ ID NO: 6 - DNA, 252 bp, Rattus norvegicus
  701918575H1
gggggcccagctggcatgctggacaaattctgaaactccatgggcgtatttccctaagatttctatctacctgagagatgagaacgccagtcgctccttccgaatcaccattctgccgcagctctacattcagcccatgatgggagctggtttcaattatgagtgctaccgttttggtatctcctcttccacaaatgcgctggtgattggtgccaccgtgatggagggattctacgtggtctttgacagagc

SEQ ID NO: 7 - DNA, 140 bp, Rattus norvegicus
  700921566H1
cgccagtcgctccttccgaatcaccattctgccgcagctctacattcagcccatgatgggagctggtttcaattatgagtgctaccgttttggtatctcctcttccacaaatgcgctggtgatggtgccaccgtgatgga

SEQ ID NO: 8 - DNA, 279 bp, Rattus norvegicus
  700494841H1
cttattctgtcctgcctttctctgccttttagtcacctacactntgctcttcctcaagccctgagatgttgacttttccgcaacccctttatcagcacacattcatttgtctttcaagttgtagtagccatgtctctttacatactttgtctgcttcttggcactccgctgcctcagcttggggacatacctgaggaaggatggtgatgcggaaggactgattggtgacttcacccatgaggtaaagtgaaatgactgggaaaatgttccaaggggtcg

SEQ ID NO: 9 - DNA, 281 bp, Rattus norvegicus
  701901708H1
ctggcagcaagacatgagcagactgaccccacaccctcctcaccatggcaagcgctgacagcaaagccaattcgctttcgggctcgatcaaagaccacatagaagccttccatgatgaccgctcccataacggtgcctgtggatgactgtgagacggcgaacttgtaacagtcgtcttgggacgtggccacatcttccactggccgtagtattgctgggggaaacaatcacaggatgagagcatgtcttattctgtcctgcctttctctgccttttagtca

SEQ ID NO: 10 - DNA, 303 bp, Rattus norvegicus
  701911752H1
caagccttagccctacctttcacacatggtggattagccagangctactacctcttgaacagacagatctgggaattagctgctggcagcaagacatgancagactgancccacaccctcctcaccatggcaagcgctgacagcaaagccaattcgctttcgggctcgatcaaagaccacatagaagcnttccatgatgaccgctcccataacggtgcctgtggatgactgtgagacggcgaattgtaacagtcgtcttgggacgtggccacatcttccactggccgtggnattgctggggga

SEQ ID NO: 11 - DNA, 284 bp, Rattus norvegicus
  701823687H1
agccgccagangtagcaactagagtccagaagggaactcttctgtgttttcattactgtacttaagctctatgctcaggttctgtgatggctctgcaggccctgcccttcaccttagggagtgctgtgtgattgccaagtttctgccttggcnagccctaacctcagcccaggtcctgtgcctccaccctctcctccaccctcatggttctctcacactgggattaacctcttactctcactgggtccccttccagtttattacctctttgcagtccatttcag

SEQ ID NO: 12 - DNA, 309 bp, Rattus norvegicus
  701596150H1
gtataagaactaacacttcgtacatcagcttcgaccttcacagtctctgaaaaagtgtgtgtatccttacctctgctttgtagatgaggagtctgggcactgcagcgaggagcccaaaggcacttagtaacagcaggactcagtcaggtcctcgcagaagccttgcccggagccgccagaggtagcaactagagtccagaagggaactcttctgtgttttcattactgtacttaagctctatgctcaggttctgtgatggctctgcaggccctgcccttcaccttagggagtgctgtgtgattgccaag

SEQ ID NO: 13 - DNA, 158 bp, Rattus norvegicus
  700542176H1
tcatgccccatgcatggtaatgtttagtttcttaaatgtgttcagaagaaacacatcagattatttgtatcaaaatctataatccttgacaggaagttatttaaacctaagccagtacaataaacgtggaatgaacaaattcaaaaaaaaaaaaangg

SEQ ID NO: 14 - DNA, 249 bp, Rattus norvegicus
  701031389H1
agtctctgaaaaagtgtgtgtatccttacctctgctttgtagatgaggagtctggcactgcagcgaggagcccaaaggcacttagtaacagcaggactcagtcaggtcctcgcagaagccttgcccggagccgccagaggtagcaactagagtccagaagggaactcttctgtgttttcattactgtacttaagctctatgctcaggttctgtgatggctctgcaggccctgcccttcaccttagggag

SEQ ID NO: 15 - DNA, 206 bp, Rattus norvegicus
  700376870H1
tttagtttcttaaatgtgttcagaagaaacacatcagattatttgtatcaaaatctataatccttgacaggaagttatttaaacctaagccagtacaataaacgtggaatgaacaaattctatgctgctttcatatcgttttgtctttgcctgttggagaaccactcattactgtcctgtcctaatcaataaatgggtttgaactg

SEQ ID NO: 16 - DNA, 91 bp, Macaca fascicularis
  700709032H1
ggcctctgtcggagggagcatgatcattggaggtatcgaccactcgctgtaacacaggcagtctctggtatacacccattcccggcgggag

SEQ ID NO: 17 - DNA, 252 bp, Homo sapiens
  5577513H1
ctgaatcagacaagttcttcatcaacggctccaactgggaaggcatcctggggctggcctatgctgagattgccaggctttgtggtgctggcttcccccncaaccagtctgaagtgctggcctctgtcggagggagcatgatcattggaggtatcgaccactcgctgtacacaggcagtcnctggtatacacccatccggcgggagtggtattatgaggtgatcattgtgcgggtggagatcaatggacagg

SEQ ID NO: 18 - DNA, 205 bp, Homo sapiens
  5094633H1
agagcattgtggacagtggcaccacatngcccaagaaagtgttngaagctgcagtcaaatccatcaaggcagcctcctccacggagaagttccctgatgggttctggctaggagagcagctggtgtgctggcaagcaggcaccaccccttggaacattttcccagtcatctcactctacctaatgggtgaggttaccaaccagtc

SEQ ID NO: 19 - DNA, 249 bp, Homo sapiens
  2293393H1
cnatgatctagggaaaaanagaggcaggtacccgtntcctggcacagaaggagagtgagtcccccaaggaccaagcaataagatcagtgatttcttggggtggcaaggtcttctacaggctacccttttcatcttcctgcttctaaacaaatcatacccaaagtgatttctagnttnttaaatgtgttcaggagaaaagactttccgggatttttanttanttgtttcngaatcatacagcccttgata

SEQ ID NO: 20 - DNA, 236 bp, Homo sapiens
  2908594H1
gtacagcgagtggtcgatacctccantgatctagggaaaaaaanaggcaggtacccgtgtcntggcacagaaggagagtgagtcccccaangaccaagcaataagatcagtgatttcttggggtggcaaggtcttctacaggctacccttttcntcttcctgcttctanacaaatcntacccaaagtgatttctagtttcttanatgtgttcangagaaaagactttccgggattt

SEQ ID NO: 21 - DNA, 165 bp, Homo sapiens
  2007269H1
gatacctccaatgatctagggaaaaaaagaggcaggtacccgtgtcctggcacagaaggagagtgagtcccccaaggaccaagcaataagatcagtgatttcttggggtggcaaggtcttctacaggctacccttttcatcttcctgcttctaaacaaatcatac

SEQ ID NO: 22 - DNA, 382 bp, Homo sapiens
  1438613F1
gacccctttactgctggacactactcatctgttttgtcttccaagttctggcaagccatacctgctcactgtctcccagtgtgtacttttaagagagatccccctgactcaggctgggacatacctgcggaaggatggtgatgcggaaggactgtccactcacggaggaggctgccttgatggatttgactgcagcttcaaacactttcttgggcaaacgaagtccattttcagatcctgtccattgatctccacccgcacaatgatcacctcataataccactcccgccggatgggtgtataccagagactgcctgtgtacagcgagtggtcgatacctccaatgatctagggaaaaaaaagaggcaggtanncgtgtcct

SEQ ID NO: 23 - DNA, 336 bp, Homo sapiens
  3484819H1
tgacacgggtacctgcctctttttttccctagatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccc

SEQ ID NO: 24 - DNA, 279 bp, Homo sapiens
  2757870H1
gaccattaagcccctgactgttctaggctcaacttccaaccctttctgcaggtcctattacctctgcctcatcctcccaacatgataaccagagtcttccttcacattgtactgcctacccccttatgttcccaggctctcccttggttttattacctccttgcagtccattttcagatcctgtccattgatctccacccgcacaatgatcacctcataataccactcccgccggatgggtgtataccagagactgcctgtgtacagcgagtggtcgat

SEQ ID NO: 25 - DNA, 305 bp, Homo sapiens
  2900063H1
ctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtg

SEQ ID NO: 26 - DNA, 302 bp, Homo sapiens
  2123411H1
ccagagtcttccttcacattgtactgcctacccccttatgttcccaggctctcccttggttttattacctccttgcagtccattttcagatcctgtccattgatctccacccgcacaatgatgacctcataataccactcccgccggatgggtgtataccagagactgcctgtgtacagcgagtggtcgatacctccaatgatctagggaaaaaaagaggcaggtacccgtgtcctggcacagaaggagagtgagtcccccaaggaccaagcaataagatcagtgatttcttggggtggcaa

SEQ ID NO: 27 - DNA, 263 bp, Homo sapiens
  1438719H1
gacccctttactgctggacactactcatctgttttgtcttccaagttctggcaagccatacctgctcactgtctcccagtgtgtacttttaagagagatccccctgactcaggctgggacatacctgcggaaggatggtgatgcggaaggactgtccactcacggaggaggctgccttgatggatttgactgcagcttcaaacactttcttgggcaaacgaagtccattttcagatcctgtccattgatctccacccgcacaa

SEQ ID NO: 28 - DNA, 255 bp, Homo sapiens
  1438613H1
gacccctttactgctggacactactcatctgttttgtcttccaagttctggcaagccatacctgctcactgtctcccagtgtgtacttttaagagagatccccctgactcaggctgggacatacctgcggaaggatggtgatgcggaaggactgtccactcacggaggaggctgccttgatggatttgactgcagcttcaaacactttcttgggcaaacgaagtccattttcagatcctgtccattgatctccac

SEQ ID NO: 29 - DNA, 476 bp, Homo sapiens
  5157179F6
ccagagataanaaagagctcagccttggtttcaagcaccaacggcnataaaaaggaaacccagttatttaacagaatccattctcttttaaggctcagtgtgtatctgagacaggaccatttcccctctccagtgcaagcttcttcatggactttgttcagatttcaattttataatagctttttctattctcctcgtcagtatactttttttttgttccaactgataaattattcttgctacttagtttnaaaaacattattcaagagtattgccttattaaaataacttgatgctttanatttttttttnctccttaaaacataaaagctacagattctcgttgacactggaagcagtaactttgcngtggcaggaaccccgtactcctacatagacacgtactttgacacagagaggtctagcacataccgctccaaggggttttgacggtaacagtggaagtacacacaagg

SEQ ID NO: 30 - DNA, 182 bp, Homo sapiens
  3985758H1
gcnacggttggcgctcgncctggagcctgccctggcgtncccccgcgggcgcagccaagcttcttggcnatggtagataactgcaggggactctggccgcggctaactancctggagatgctgatcgggacccccccgcagaagctacagattctcgttgacantggaagcagtaactttga

SEQ ID NO: 31 - DNA, 256 bp, Homo sapiens
  2846604H1
aagagtattgccttattaaaataacttgatgctttctattttttttttctccttaaaacataaaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaag

SEQ ID NO: 32 - DNA, 226 bp, Homo sapiens
  5743028H1
aacacatcacgcaccttttgggtgtctaccctggtaccgcctttcttttcaagagaccattcttcaacagaactgtaaggtttcttcttggctgaatcagatgtgacgcatcccacttctgcgtttgaggtctagcacataccgctccaagggctttgacgtcacagtgaagtacacacaaggaagctggacgggcttcgttggggaagacctcgtcaccatcccc

SEQ ID NO: 33 - DNA, 259 bp, Homo sapiens
  3751907H1
gggactgggtcccagctggcgtgctggacgaattcggaaacaccttggtcttacttccctaaaatctccatctacctgagagatgagaactccagcaggtcattccgtatcacaatcctgcctcaggtatgaacttggatttgtgctttgctctttttatcatgcaaaagagaaagcactccatgcatgacacgtgtatagatgtcacacctgcattagatctctatcaccaccataatgctgcaaaaggacagccaca

SEQ ID NO: 34 - DNA, 263 bp, Homo sapiens
  4148749H1
attgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttnatgcggtggtggaagctgtggcccgcgcatctctgattccagaattcnctgatggtttctggactgggtccca

SEQ ID NO: 35 - DNA, 262 bp, Homo sapiens
  1855226H1
gagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattc

SEQ ID NO: 36 - DNA, 268 bp, Homo sapiens
  1855420H1
gagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaac

SEQ ID NO: 37 - DNA, 257 bp, Homo sapiens
  5114558H1
agacatctggtatacncctattaaggaagagtggtactaccagatagaaattctgacattggaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaatctctgatggtttctggactgggtcccagctggcgtgctggacgatt

SEQ ID NO: 38 - DNA, 430 bp, Homo sapiens
  839538R1
cntctggtatanncctattaaggaagagtggtactaccagatagaaattctgaaattggaaattggaggnnaaagccttaatctggactgcagagagtataacgcagacaaggccatcgtggacagtggcaccacgctgctgcgcctgcnccagaaggtgtttgatgcggtggtggaagctgtggcccgcgcatctctgattccaganttctctgatggtttctggactgggtcccagctggcgtgctggacgaattcggaaacaccttggtcttacttccctanaatctccatctacntgagagacgagaactccagcaggtcattccgtatcacaatcctgcctcagctttacattcagcccatgatggggggnnggcctgaattatgaatgttaccgattcgcatttcncattcacnaatgcgctgg

SEQ ID NO: 39 - DNA, 217 bp, Homo sapiens
  5588490H1
ctggtatacccctattaaggaagagtggtactaccagatagaaattctngaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatgg

SEQ ID NO: 40 - DNA, 262 bp, Homo sapiens
  4999662H1
cncgnatacccctattaaggaagagtggtactaccagntacnaantctgaaattggaaattggaggccaaagccttaanctggactgcagagagtanaacgcagacanggccatcgtngacagtggcaccacgctgctgcgcctgccccagaaggtntttgatgcggtggtggaagctgtggcccgcgcatctctgattccagaattctctnatggtttctggactnggtcccanctggcgtgctggacgaattcggaaaca

SEQ ID NO: 41 - DNA, 273 bp, Homo sapiens
  3630054H1
tggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtntgatgcggtggtggaagctgtggcccgcgcatctctgattccagaattctctgatggtttctggactgggtcccagctggcgtgctggacgaattcggaaacaccttggtcttac

SEQ ID NO: 42 - DNA, 272 bp, Homo sapiens
  4108264H1
gtggtactaccagataganattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctgggacgaattncggaaacaccttggtcntacttccctannancnccatcta

SEQ ID NO: 43 - DNA, 1601 bp, Homo sapiens
  1611565CB1
agccttaatctggactgcagagagtataacgcagacaaggccatcgtggacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagacgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatgaatagccaggcctgacctcaagcaaccatgaactcagctattaagaaaatcacatttccagggcagcagccgggatcgatggtggcgctttctcctgtgcccacccgtcttcaatctctgttctgctcccagatgccttctagattcactgtcttttgattcttgattttcaagctttcaaatcctccctacttccaagaaaaataattaaaaaaaaaacttcattctaaaccaaaaaaa

SEQ ID NO: 44 - DNA, 1911 bp, Homo sapiens
  1869869
atacgcctcacatagggaatttggccctcgaggcaagaattcggcagagctcagtgccttaagatggtctttcccaaggagcatcccccaagccttcaaatttgccttccagtttagacaggaagctggcctctgcctttagggccgtatgaggactcactccttgtctctttctgccaaagcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatggggtttgtttttcaatggtctcttttccgtggtggcagccggaccaaaagtgagatggggaagggcctatctagcc

SEQ ID NO: 45 - DNA, 1366 bp, Homo sapiens
  g1658285
cgcagaactcagagctgctcttcctctgtggccagttggggaccagcatcatgaagtggatggtggtggtcttggtctgcctccagctcttggaggcagcagtggtcaaagtgcccctgaagaaatttaagtctatccgtgagaccatgaaggagaagggcttgctgggggagttcctgaggacccacaagtatgatcctgcttggaagtaccgctttggtgacctcagcgtgacctacgagcccatggcctacatggatgctgcctactttggtgagatcagcatcgggactccaccccagaacttcctggtcctttttgacaccggctcctccaacttgtgggtgccctctgtctactgccagagccaggcctgcaccagtcactcccgcttcaaccccagcgagtcgtccacctactccaccaatgggcagaccttctccctgcagtatggcagtggcagcctcaccggcttctttggctatgacaccctgactgtccagagcatccaggtccccaaccaggagttcggcttgagtgagaatgagcctggtaccaacttcgtctatgcgcagtttgatggcatcatgggcctggcctaccctgctctgtccgtggatgaggccaccacagctatgcagggcatggtgcaggagggcgccctcaccagccccgtcttcagcgtctacctcagcaaccagcagggctccagcgggggagcggttgtctttgggggtgtggatagcagcctgtacacggggcagatctactgggcgcctgtcacccaggaactctactggcagattggcattgaagagttcctcatcggcggccaggcctccggctggtgttctgagggttgccaggccatcgtggacacaggcacctctctgctaactgtgccccagcagtacatgagtgctcttctgcaggccacaggggcccaggaggatgagtatggacagtttctcgtgaactgtaacagcattcagaatctgcccagcttgaccttcatcatcaatggtgtggagttccctctgccaccttcctcctatatcctcagtaacaacggctactgcaccgtgggagtcgagcccacctacctgtcctcccagaacggccagcccctgtggatcctcggggatgtcttcctcaggtcctactattccgtctacgacttgggcaacaacagagtaggctttgccactgccgcctagacttgctgcctcgacacgtgggctcccctcttcctcttgaccctgcaccctcctagggcattgtatctgtctttccactctggattcagccttctttttctggactctggactttctctaataataaatagttcttctaaaaaaaaaaa

SEQ ID NO: 46 - protein, 423 aa, Homo sapiens
  1611565CD1
MLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 47 - protein, 322 aa, Homo sapiens
  1869868
GLTPCLFLPKPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 48 - protein, 388 aa, Homo sapiens
  g1658286
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAA

SEQ ID NO: 49 - protein, 396 aa, Neurospora crassa
  g1039445
MKGALLTAAMLLGSAQAGVHTMKLKKVPLADELESVPIDVQVQHLGQKYTGLRTESHTQAMFKATDAQVSGNHPVPITNFMNAQYFSEITIGTPPQTFKVVLDTGSSNLWVPSSQCGSIACYLHNKYESSESSTYKKNGTSFKIEYGSGSLSGFVSQDRMTIGDITINDQLFAEATSEPGLAFAFGRFDGILGLGYDRLAVPGITPPFYKMVEQKLVDEPVFSFYLADQDGESEVVFGGVNKDRYTGKITTIPLRRKAYWEVDFDAIGYGKDFAELEGHGVILDTGTSLIALPSQLAEMLNAQIGAKKSWNGQFTIDCGKKSSLEDVTFTLAGYNFTLGPEDYILEASGSCLSTFMGMDMPAPVGPLAILGDAFLRKYYSIYDLGADTVGIATAKR

(19) United States
(12) Patent Application Publication
(10) Pub. No.: US 2002/0169304 A1
(43) Pub. Date: Nov. 14, 2002

(54) ASPARTYL PROTEASES

(76) Inventors: MATTHEW R. KASER, CASTRO VALLEY, CA (US); Olga Bandman, Mountain view, CA (US); Jennifer L. Hillman, Mountain view, CA (US); Preeti Lal, Santa clara, CA (US); BENJAMIN G. COCKS, SUNNYVALE, CA (US); JEANNE LORING, FORSTER CITY, CA (US); Y. TOM TANG, SAN JOSE, CA (US); HENRY YUE, SUNNYVALE, CA (US)

Correspondence Address: INCYTE GENOMICS, INC., 3160 PORTER DRIVE, PALO ALTO, CA, 94304, US

(21) Appl. No.: 09/470,954

(22) Filed: Dec. 22, 1999

(51) Int. Cl. C12Q 1/68; C07H 21/04; C12N 15/09; C12N 15/63; C12N 15/70; C12N 1/20; C12N 15/74

(52) U.S. Cl. 536/23.2; 435/320.1; 435/252.3

(57) ABSTRACT

The invention provide mammalian nucleic acid molecules and fragments thereof. It also provides for the use of the mammalian nucleic acid molecules for the characterization, diagnosis, evaluation, treatment, or prevention of conditions, diseases and disorders associated with Alzheimer's disease and Down syndrome. The invention additionally provides expression vectors and host cells for the production of the protein encoded by the mammalian nucleic acid molecules.

### FIELD OF THE INVENTION

This invention relates to nucleic acid and amino acid sequences of new mammalian protein, nucleic acid sequence variants thereof, and to the use of these sequences in the characterization, diagnosis, prevention, and treatment of Alzheimer's disease and Down syndrome.

### BACKGROUND OF THE INVENTION

Phylogenetic relationships among organisms have been demonstrated many times, and studies from a diversity of prokaryotic and eukaryotic organisms suggest a more or less gradual evolution of biochemical and physiological mechanisms and metabolic pathways. Despite different evolutionary pressures, proteins that regulate the cell cycle in yeast, nematode, fly, rat, and man have common chemical or structural features and modulate the same general cellular activity. Comparisons of human gene sequences with those from other organisms where the structure and/or function may be known allow researchers to draw analogies and to develop model systems for testing hypotheses. These model systems are of great importance in developing and testing diagnostic and therapeutic agents for human conditions, diseases and disorders.

Senile neuritic plaques, the neuropathological signature of Alzheimer's disease at post-mortem examination, are found in the hippocampus and neocortical regions of the brain. The hippocampus is part of the brain's limbic system and plays an important role in learning and memory. In Alzheimer's disease patients, accumulating plaques damage the neuronal architecture of the hippocampus and eventually cripple the memory process. The disease can be early onset, effecting individuals as young as 40 years of age, and can be familial or sporadic. Familial Alzheimer's disease has been thought to be inherited strictly as an autosomal dominant trait. However, this view is changing as more genetic determinants are isolated. For example, some normal allele variants of apolipoprotein E (ApoE), a protein found in senile plaques, can either protect against, or increase, the risk of developing the disease (Strittmatter et al. (1993) Proc. Natl. Acad. Sci. 90:1977-1981). Mutations in three other genes, amyloid precursor protein (APP), presenilin-1, and presenilin-2, have also been shown to predispose an individual to Alzheimer's disease (Selkoe (1999) Nature 399:A23-A31).

β-amyloid protein (Aβ) is the major component of senile plaques, and it is normally formed when β- and γ-secretases cleave APP. In Alzheimer's disease patients, large quantities of Aβ are generated and accumulate extracellularly in these neuropathological plaques. Efforts to understand the mechanism underlying Aβ deposition have recently focused on the APP-cleaving secretases since two yeast aspartyl proteases have been shown to process APP in vitro (Zhang et al. (1997) Biochim Biophys Acta 1359:110-122). Recent evidence using peptidomimetic probes further confirms that the secretases are intramembrane-cleaving aspartyl proteases (Wolfe et al. (1999) Biochemistry 38:4720-4727).

The presenilin-1 gene is a candidate for the γ-secretase that cleaves the APP carboxyl terminus. Several lines of evidence support the involvement of presenilins in the disease process. These include coimmunoprecipitation of presenilin and APP and discovery that missense point mutations in presenilin genes can result in a particularly aggressive, early onset form of the disease (Haas and De Strooper (1999) Science 286:916-919). Presenilins are thought to cleave notch protein, which, like APP, is cleaved by α- and β-secretases in its ectodomain to produce the carboxyl terminal fragment that is a substrate for γ-secretases (Haas and De Strooper, supra).

Aspartic proteases are members of the cathepsin family of lysosomal proteases and include pepsin A, gastricsin, chymosin, renin, and cathepsins D and E. Aspartic proteases include two aspartic acid residues in the active site and are most active between pH 2-3, where one of the aspartic acid residues is ionized and the other not. The catalytic aspartic acid residue of the aspartyl protease active site is characterized by the amino acid residue sequence D-T/S-G-T/S-T/S (aspartyl protease signature PS00141; PROSITE PDOC00128). A potent inhibitor of aspartic proteases is the hexapeptide pepstatin which, in the transition state, resembles normal substrates. Aspartic proteases also include bacterial penicillopepsin, mammalian pepsin and chymosin, and certain fungal proteases. Abnormal regulation and expression of cathepsins is evident in various inflammatory disease states such as arthritis.

The discovery of additional nucleic acid sequences encoding aspartyl proteases and nucleic acid sequence variants thereof provides new compositions that are useful in the characterization, diagnosis, prevention, and treatment of Alzheimer's disease and Down syndrome.

### SUMMARY OF THE INVENTION

The invention is based on the discovery of mammalian nucleic acid molecules encoding aspartyl protease (ASP) and nucleic acid sequence variants thereof, which satisfies a need in the art by providing compositions useful in the characterization, diagnosis, prevention, and treatment of conditions such as Alzheimer's disease and Down syndrome.

The invention provides isolated and purified human, monkey, and rat nucleic acid molecules comprising SEQ ID NOs: 1-43, and fragments thereof, encoding the mammalian protein comprising amino acid sequences of SEQ ID NOs:46 and 47 or portions thereof, a biologically active fragment of SEQ ID NO:46, and an immunologically active fragment of SEQ ID NO:46.

The invention further provides a probe that hybridizes under high stringency conditions to the mammalian nucleic acid molecule or fragments thereof. The invention also provides isolated and purified nucleic acid molecules that are complementary to the nucleic acid molecules of SEQ ID NOs: 1-43. In one aspect, the probe is a single stranded complementary RNA or DNA molecule.

The invention further provides a method for detecting a nucleic acid molecule in a sample, the method comprising the steps of hybridizing a probe to at least one nucleic acid molecule of a sample, forming a hybridization complex; and detecting the hybridization complex, wherein the presence of the hybridization complex indicates the presence of the nucleic acid molecule in the sample. In one aspect, the method further comprises amplifying the nucleic acid molecule prior to hybridization. The nucleic acid molecule or a fragment thereof may comprise either an element or a target on a microarray.

The invention also provides a method for using a nucleic acid molecule or a fragment thereof to screen a library of molecules to identify at least one ligand that specifically binds the nucleic acid molecule, the method comprising combining the nucleic acid molecule with a library of molecules under conditions allowing specific binding, and detecting specific binding, thereby identifying a ligand that specifically binds the nucleic acid molecule. Such libraries include DNA and RNA molecules, peptides, PNAs, proteins, and the like. In an analogous method, the nucleic acid molecule or a fragment thereof is used to purify a ligand.

The invention also provides an expression vector containing at least a fragment of the nucleic acid molecule. In another aspect, the expression vector is contained within a host cell. The invention further provides a method for producing a protein, the method comprising the steps of culturing the host cell under conditions for the expression of the protein and recovering the protein from the host cell culture.

The invention also provides substantially purified mammalian ASP or a portion thereof. The invention further provides isolated and purified proteins having the amino acid sequence of SEQ ID NO:46, a biologically active fragment of SEQ ID NO:46, and an immunologically active fragment of SEQ ID NO:46. Additionally, the invention provides a pharmaceutical composition comprising a substantially purified mammalian protein or a portion thereof in conjunction with a pharmaceutical carrier.

The invention further provides a method for using at least a portion of the mammalian protein to produce antibodies. The invention also provides a method for using a mammalian protein or a portion thereof to screen a library of molecules to identify at least one ligand that specifically binds the protein, the method comprising combining the protein with the library of molecules under conditions allowing specific binding, and detecting specific binding, thereby identifying a ligand that specifically binds the protein. Such libraries include DNA and RNA molecules, peptides, agonists, antagonists, antibodies, immunoglobulins, drug compounds, pharmaceutical agents, and other ligands. In one aspect, the ligand identified using the method modulates the activity of the mammalian protein. In an analogous method, the protein or a portion thereof is used to purify a ligand. The method involves combining the mammalian protein or a portion thereof with a sample under conditions to allow specific binding, detecting specific binding between the protein and ligand, recovering the bound protein, and separating the protein from the ligand to obtain purified ligand.

The invention further provides a method for inserting a marker gene into the genomic DNA of a mammal to disrupt the expression of the natural mammalian nucleic acid molecule. The invention also provides a method for using the mammalian nucleic acid molecule to produce a mammalian model system, the method comprising constructing a vector containing the mammalian nucleic acid molecule; introducing the vector into a totipotent mammalian embryonic stem cell; selecting an embryonic stem cell with the vector integrated into genomic DNA; microinjecting the selected cell into a mammalian blastocyst, thereby forming a chimeric blastocyst; transferring the chimeric blastocyst into a pseudopregnant dam, wherein the dam gives birth to a chimeric mammal containing at least one additional copy of mammalian nucleic acid molecule in its germ line; and breeding the chimeric mammal to generate a homozygous mammalian model system.

### BRIEF DESCRIPTION OF THE FIGURES AND TABLE

FIGS. 1A, 1B, 1C, 1D, 1E, and 1F show the human nucleic acid molecule (SEQ ID NO:44) encoding the human amino acid sequence (SEQ ID NO:47) of the mammalian protein. The alignment was produced using MACDNASIS PRO software (Hitachi Software Engineering, South San Francisco Calif.).

FIGS. 2A, 2B, 2C, 2D, 2E, and 2F show the human nucleic acid molecule (SEQ ID NO:43) encoding the human amino acid sequence (SEQ ID NO:46) of the mammalian protein. The alignment was produced using MACDNASIS PRO software (Hitachi Software Engineering)

FIGS. 3A, 3B, 3C, and 3D demonstrate the chemical and structural similarity among human ASP1, clone ID 1869869 (SEQ ID NO:47), human ASP2, clone ID 1611565 (SEQ ID NO:46), human gastricsin, GI 1658286 (SEQ ID NO:48), Neurospora crassa vacuolar protease, GI 1039445 (SEQ ID NO:49) proteins, produced using the MEGALIGN program of LASERGENE software (DNASTAR, Madison Wis.). Conserved aspartyl protease active sites (D-S/T-G-S/T-S/T) are double underlined.

FIGS. 4A, 4B, 4C, 4D, 4E, 4F, 4G, and 4H demonstrate the chemical and structural similarity among the nucleic acids encoding human ASP1, clone ID 1869869 (SEQ ID NO:44) and nucleic acid variants, 1438613F1 (SEQ ID NO:22), 1438719H1 (SEQ ID NO:27), 1438613H1 (SEQ ID NO:28), 5094633H1 (SEQ ID NO:18), 2900063H1 (SEQ ID NO:25), 3484819H1 (SEQ ID NO:23), 2757870H1 (SEQ ID NO:24), 212341H1 (SEQ ID NO:26), 5577513H1 (SEQ ID NO:17), 2908594H1 (SEQ ID NO:20), 2007269H1 (SEQ ID NO:21), and 2293393H1 (SEQ ID NO: 19), produced using the SEQMAN II program of LASERGENE software (DNASTAR). Putative splice junction sites are indicated by (Δ).

FIGS. 5A, 5B, 5C, 5D, 5E, 5F and 5G demonstrate the chemical and structural similarity among the nucleic acids encoding human ASP2, 1611565CB1 (SEQ ID NO:43) and nucleic acid variants, 5157179F6 (SEQ ID NO:29), 3985758H1 (SEQ ID NO:30), 2846604H1 (SEQ ID NO:31), 5743028H1 (SEQ ID NO:32), 4148749H1 (SEQ ID NO:34), 1855226H1 (SEQ ID NO:35), 1855420H1 (SEQ ID NO:36), 5114558H1 (SEQ ID NO:37), 839538R1 (SEQ ID NO:38), 4999662H1 (SEQ ID NO:40), 5588490H1 (SEQ ID NO:39), 3630054H1 (SEQ ID NO:41), 4108264H1 (SEQ ID NO:42), and 3751907H1 (SEQ ID NO:33), produced using the SEQMAN II program of LASERGENE software (DNASTAR). Putative splice junction sites are indicated by (Δ).

FIGS. 6A, 6B, 6C, 6D, and 6E demonstrate the chemical and structural similarity among human, 1611565.comp (SEQ ID NO:43); and rat, 701918575H1 (SEQ ID NO:6) and 700921566H1 (SEQ ID NO:7), nucleic acid sequences, produced using Phrap software (Phil Green, University of Washington, Seattle Wach.).

FIGS. 7A, 7B, 7C, 7D, 7E, 7F, and 7G demonstrate the chemical and structural similarity among human, 1869569.comp (SEQ ID NO:44); rat, 701911752H1 (SEQ ID NO:10), 70190708H1 (SEQ ID NO:9), 700494841H1 (SEQ ID NO:8), 701596150H1 (SEQ ID NO:12), 701031389H1 (SEQ ID NO:14) 701823687H1 (SEQ ID NO:11), 701188357H1 (SEQ ID NO:5), 700769257H1 (SEQ ID NO:4), 70076987H1 (SEQ ID NO:3), 700251567H1.comp (SEQ ID NO: 1), 701432228T1.comp (SEQ ID NO:2), 700542176H1 (SEQ ID NO:13), and 700376870H1 (SEQ ID NO:15); and monkey, 700709032H1.comp (SEQ ID NO:16), nucleic acid sequences, produced using Phrap software (Green, supra). Putative splice junction sites are indicated by (Δ).

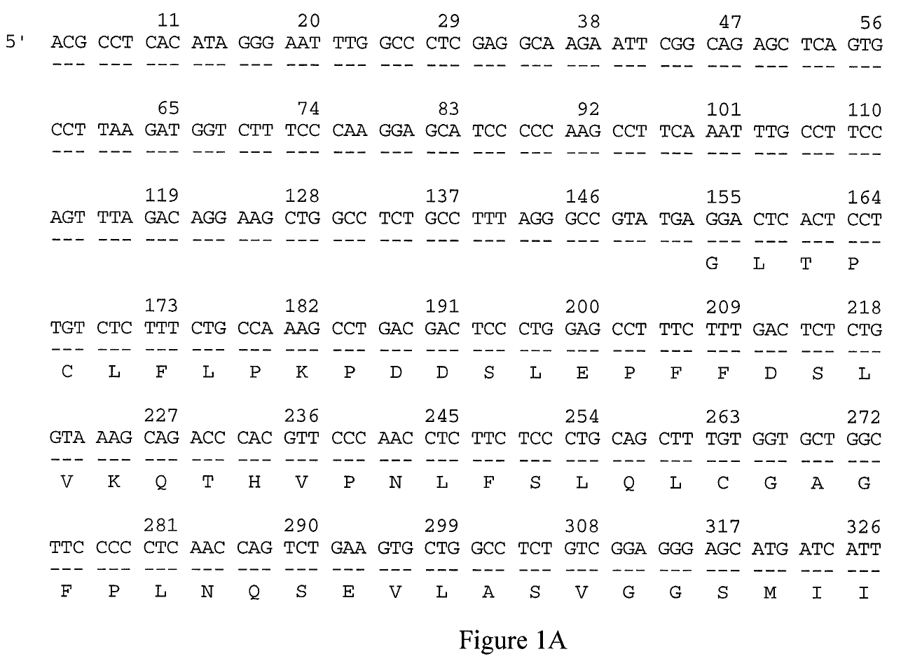

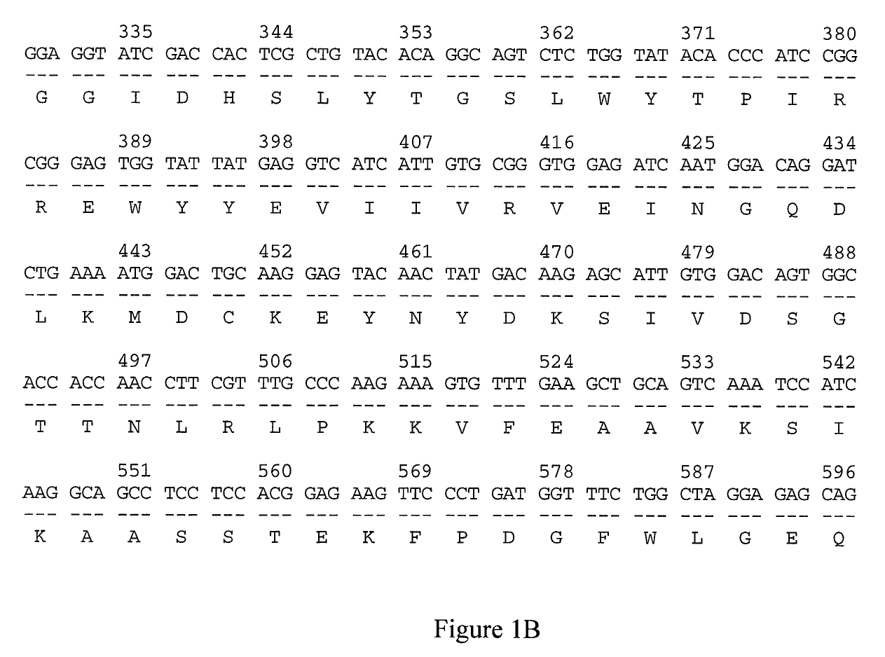

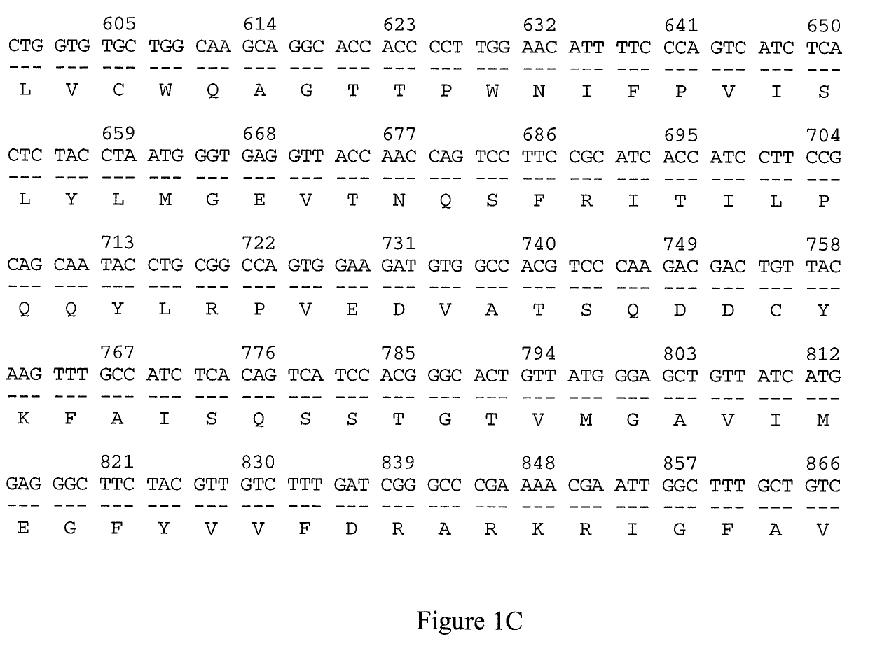

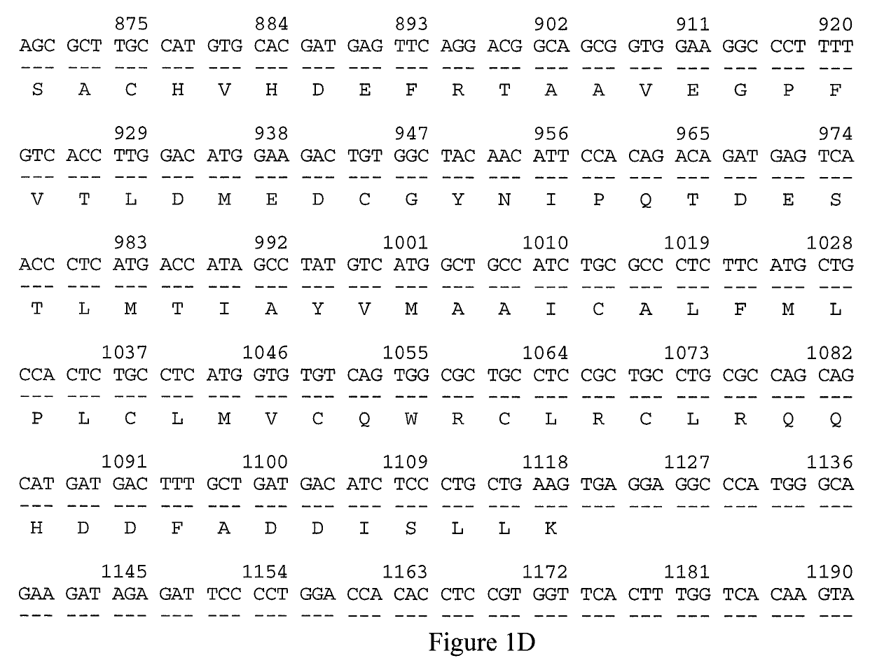

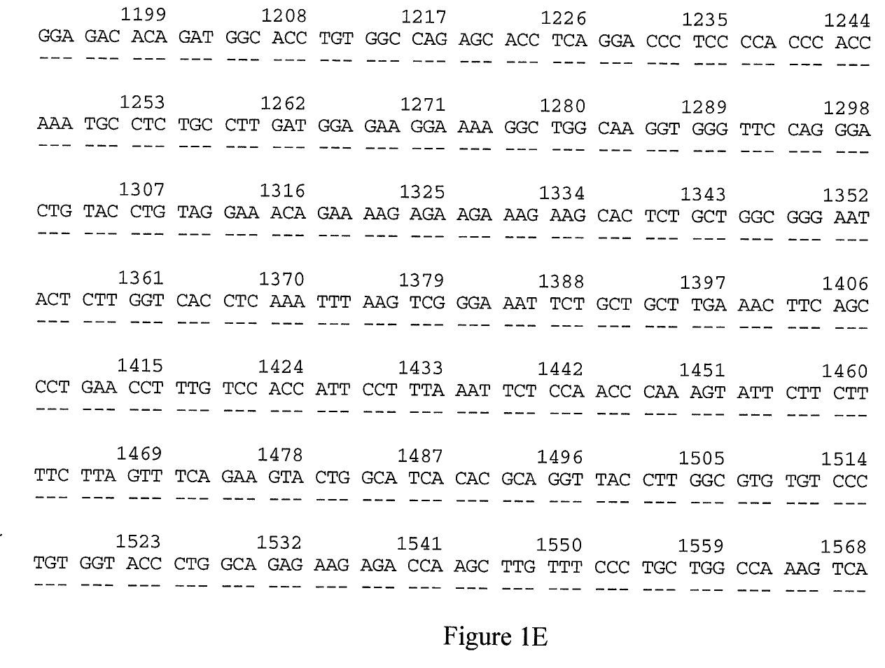

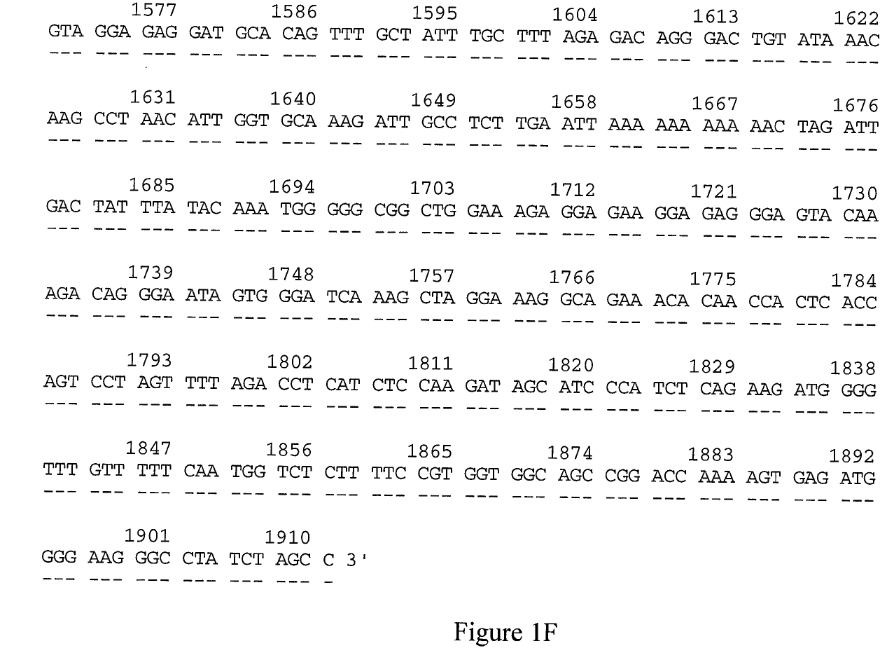

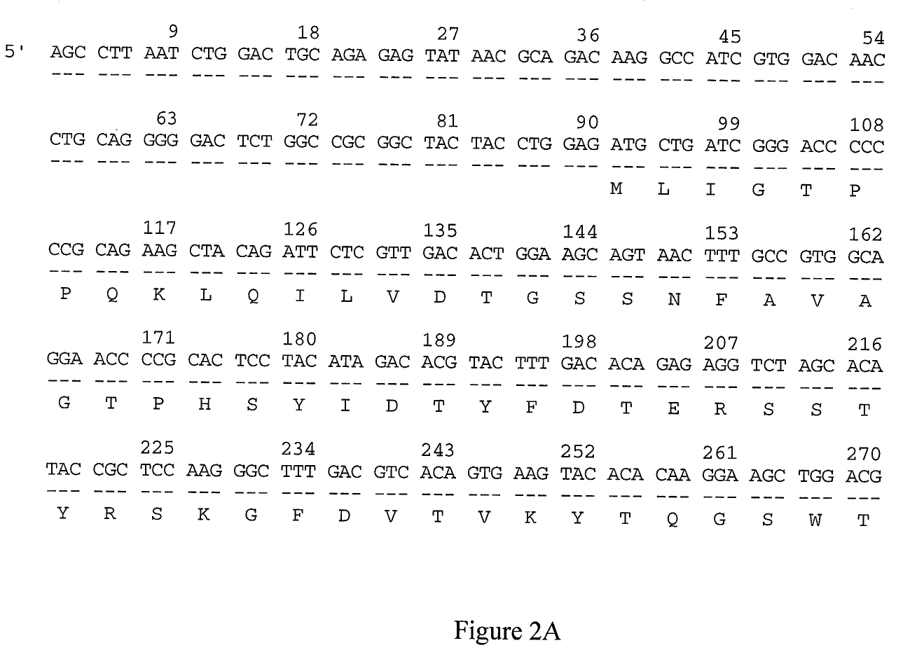

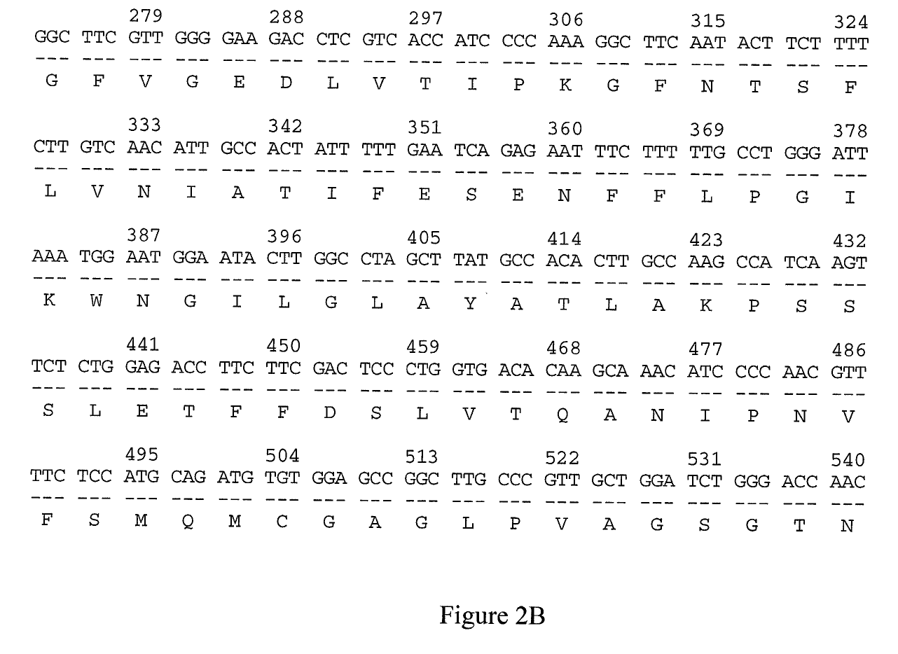

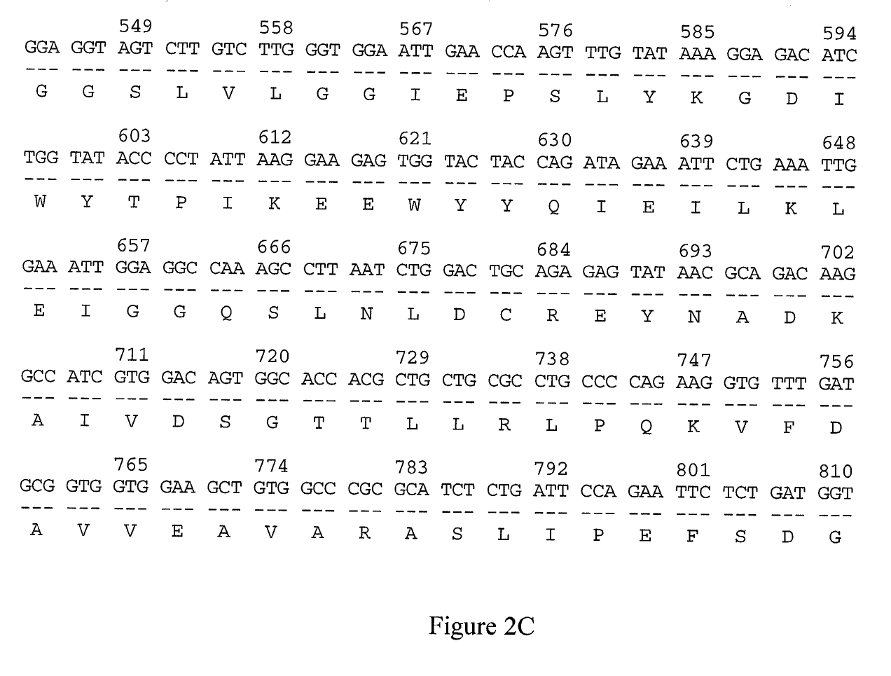

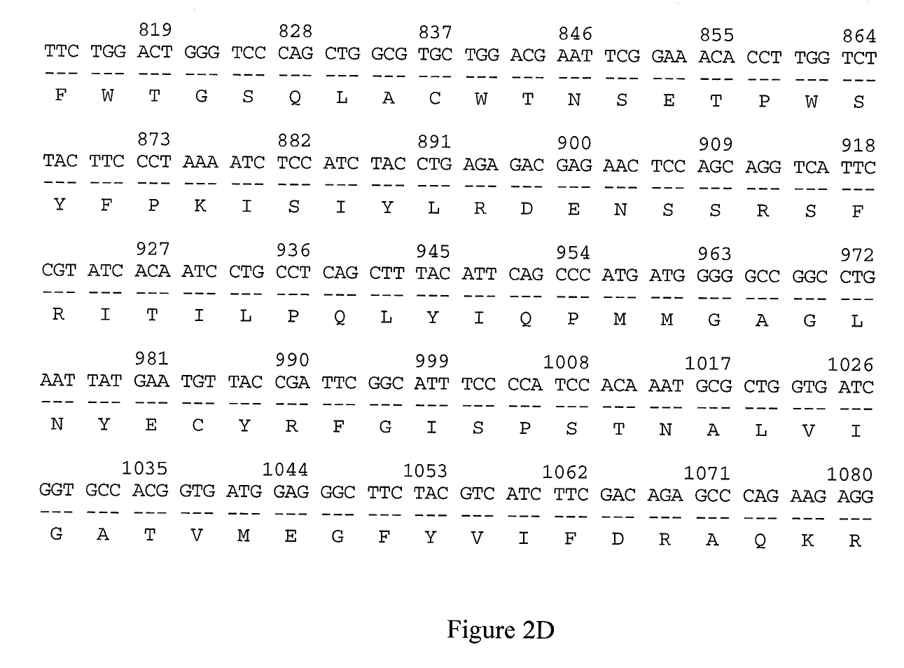

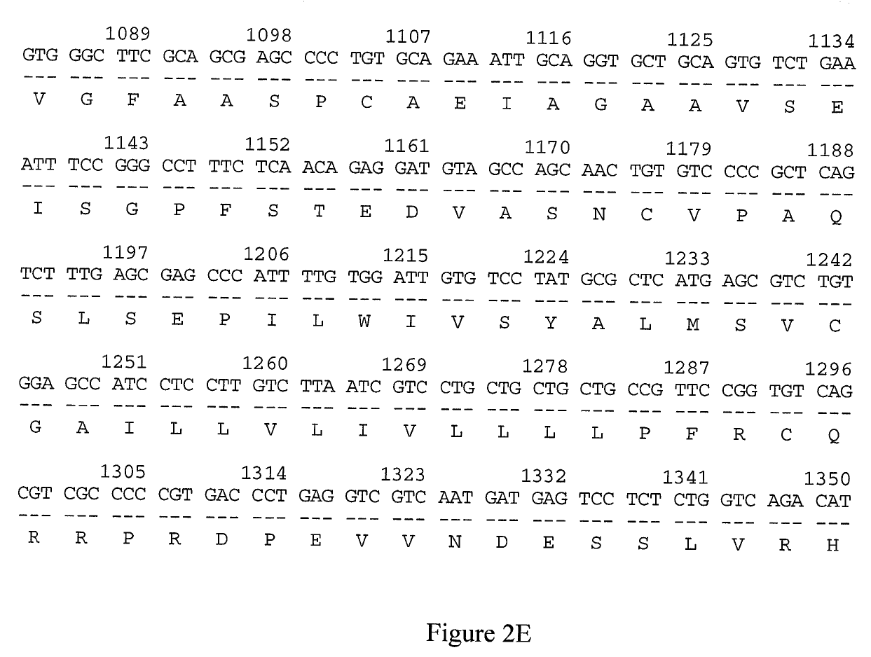

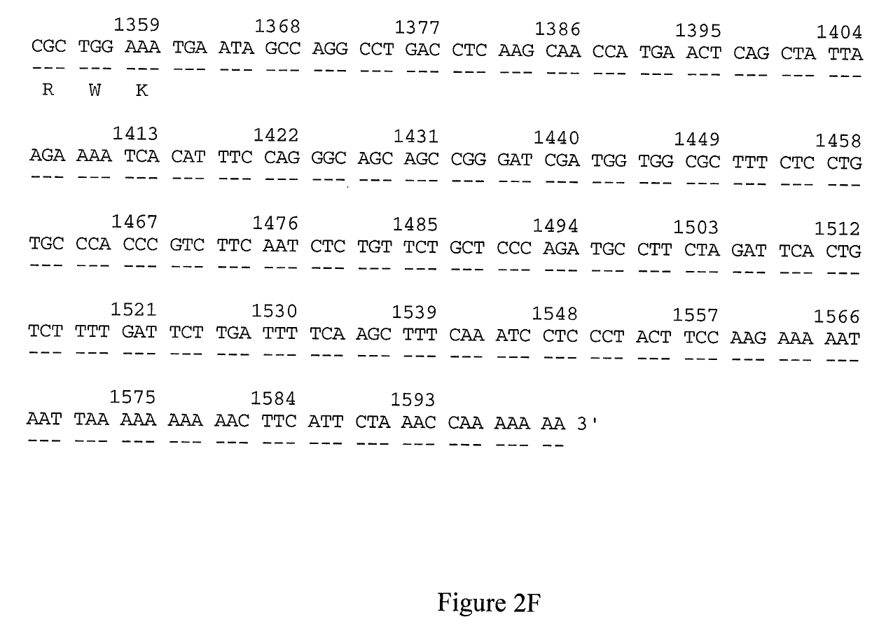

### DESCRIPTION OF THE INVENTION

It is understood that this invention is not limited to the particular machines, materials and methods described. It is also to be understood that the terminology used herein is for the purpose of describing particular embodiments only and is not intended to limit the scope of the present invention which will be limited only by the appended claims. As used herein, the singular forms “a,” “an,” and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### Definitions

“Aspartyl protease” or “ASP”, refers to a substantially purified protein obtained from any mammalian species, including murine, bovine, ovine, porcine, rodent, canine, simian, and preferably the human species, and from any source, whether natural, synthetic, semi-synthetic, or recombinant. “ASPs” refers to more than one ASP.

“Altered gene expression” and “altered expression” refers to the increase or decrease of gene expression and the presence or absence of transcribed messenger RNA transcribed in a sample.

“Biologically active” refers to a protein having structural, immunological, regulatory, or chemical functions of a naturally occurring, recombinant or synthetic molecule.

“Complementary” refer to the natural hydrogen bonding by base pairing between purines and pyrimidines. For example, the sequence A-C-G-T forms hydrogen bonds with its complements T-G-C-A or U-G-C-A. Two single-stranded molecules may be considered partially complementary, if only some of the nucleotides bond, or completely complementary, if nearly all of the nucleotides bond. The degree of complementarity between nucleic acid strands affects the efficiency and strength of the hybridization and amplification reactions.

“Derivative” refers to the chemical modification of a nucleic acid molecule or amino acid sequence. Chemical modifications can include replacement of hydrogen by an alkyl, acyl, or amino group or glycosylation, pegylation, or any similar process that retains or enhances biological activity or lifespan of the molecule or sequence.

“Fragment” refers to an Incyte clone or any part of a nucleic acid molecule that retains a usable, functional characteristic. Useful fragments include oligonucleotides which may be used in hybridization or amplification technologies or in regulation of replication, transcription or translation.

“Hybridization complex” refers to a complex between two nucleic acid molecules by virtue of the formation of hydrogen bonds between purines and pyrimidines.

“Ligand” refers to any molecule, agent, or compound that will bind specifically to a complementary site on a nucleic acid molecule or protein. Such ligands stabilize or modulate the activity of nucleic acid molecules or proteins of the invention and may be composed of at least one of the following: inorganic and organic substances including nucleic acids, proteins, carbohydrates, fats, and lipids.

“Modulates” refers to a change in activity (biological, chemical, or immunological) or lifespan resulting from specific binding between a molecule and either a nucleic acid molecule or a protein.

“Nucleic acid molecule” refers to a nucleic acid, oligonucleotide, nucleotide, polynucleotide or any fragment thereof. It may be DNA or RNA of genomic or synthetic origin, double-stranded or single-stranded, and combined with carbohydrate, lipids, protein or other materials to perform a particular activity such as transformation or form a useful composition such as a peptide nucleic acid (PNA). “Oligonucleotide” is substantially equivalent to the terms amplimer, primer, oligomer, element, target, and probe and is preferably single stranded. The nucleic acid molecules may be splice variants of another nucleic acid molecule.

“Protein” refers to an amino acid sequence, oligopeptide, peptide, polypeptide or portions thereof whether naturally occurring or synthetic.

“Portion”, as used herein, refers to any part of a protein used for any purpose, but especially for the screening of a library of molecules that specifically bind to that portion, or for the production of antibodies. An exemplary portion is the first 40 contiguous amino acids of SEQ ID NO:46.

“Similarity” as applied to polynucleotide sequences, refers to the quantified residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

A suite of commonly used and freely available sequence comparison algorithms is provided by the National Center for Biotechnology Information (NCBI), Bethesda Md. BLAST, Basic Local Alignment Search Tool (Altschul et al. (1990) J. Mol. Biol. 215:403-410; Altschul (1993) J. Mol. Evol. 36:290-300), is available from NCBI (http://www.ncbi.nlm.nih.gov/BLAST/) and several other sources. The BLAST software suite includes various sequence analysis programs including “blastn”, that is used to align a known polynucleotide sequence with other polynucleotide sequences from a variety of databases. Also available is a tool called “BLAST 2 sequences” that is used for direct pairwise comparison of two nucleotide sequences. “BLAST 2 sequences” can be accessed and used interactively at http://www.ncbi.nlm.nih.gov/gorf/b12.html. The “BLAST 2 sequences” tool can be used for both blastn and blastp. BLAST programs are commonly used with gap and other parameters set to default settings. For example, to compare two nucleotide sequences, one may use blastn with the “BLAST 2 Sequences” tool Version 2.0.9 (May 7, 1999) set at default parameters. Such default parameters may be, for example: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on.

Similarity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such length are exemplary only, and it is understood that any fragment length supported by the sequences shown herein, in the tables, figures, or Sequence Listing, may be used to describe a length over which percentage identity may be measured.

“Sample” is used in its broadest sense. A sample containing nucleic acid molecules may comprise a bodily fluid; an extract from a cell, chromosome, organelle, or membrane isolated from a cell; genomic DNA, RNA, or cDNA in solution or bound to a substrate; a cell; a tissue; a tissue print or fingerprint; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Variant” refers to nucleic acid molecules that are splice variants of a nucleic acid molecule that encodes ASP and which may be determined by BLAST score, wherein the score is at least 100, and most preferably at least 400.

### The Invention

The invention is based on the discovery of new mammalian nucleic acid molecules that encode ASPs and nucleic acid molecule variants thereof, and on the use of the nucleic acid molecules, or fragments thereof, and protein, or portions thereof, as compositions in the characterization, diagnosis, treatment, or prevention of conditions such as Alzheimer's disease and Down syndrome.

Nucleic acids encoding ASPI of the present invention were first identified in Incyte Clone 1611565H1 from the colon tissue cDNA library (COLNTUT06) using a computer search for amino acid sequence alignments. A consensus sequence, SEQ ID NO:43, was derived from Incyte Clones 1863920X321F1 (PROSNOT19), 2233901H1 (PANCTUT02), 1611565T1 (COLNTUT06), 336541H1 (EOSIHET02), and the shotgun sequence SBAA03902F1.

Nucleic acids encoding the ASP2 were first identified in Incyte Clone 1869869H1 from the skin tissue cDNA library (SKINBIT01) using a computer search for amino acid sequence alignments. A consensus sequence, SEQ ID NO:44, was derived from Incyte Clones 1869869F6 (SKINBIT01), 2549562F6 (LUNGTUT06), 3338603H1 (SPLNNOT10), and the shotgun sequences 70317925D1, 70317570D1, and 70319214D1.

Nucleic acid molecules encoding the mammalian aspartyl protease and nucleic acid molecule variants of the present invention were first identified by comparing nucleic acid sequences and amino acid sequences encoding ASP1 of the present invention and ASP2 with nucleic acid sequences in the LIFESEQ and ZOOSEQ databases (Incyte Pharmaceuticals, Palo Alto Calif.).

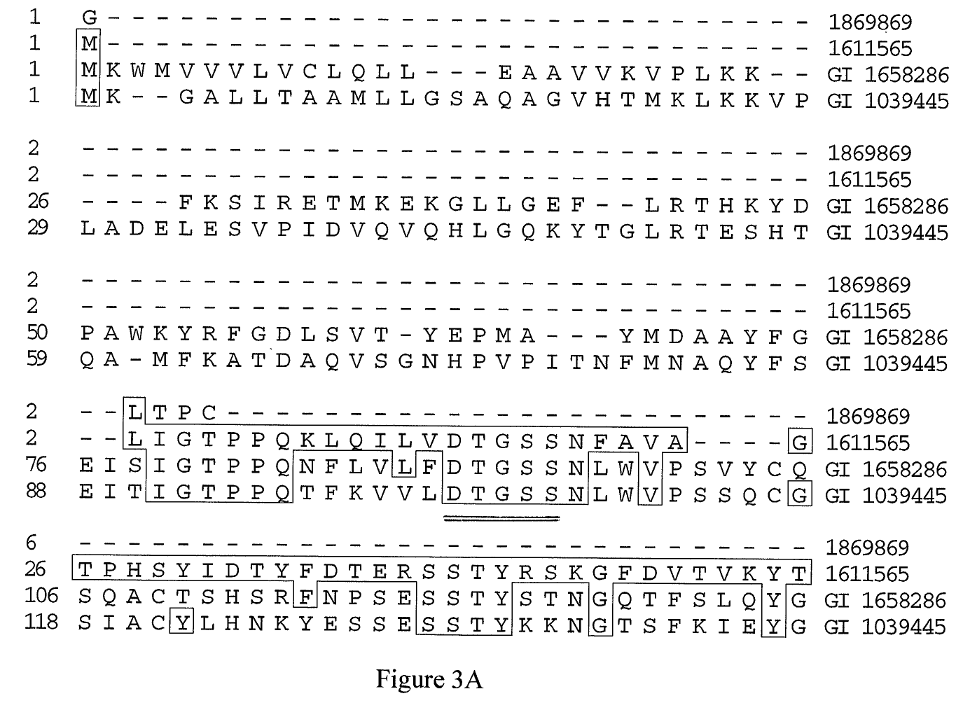

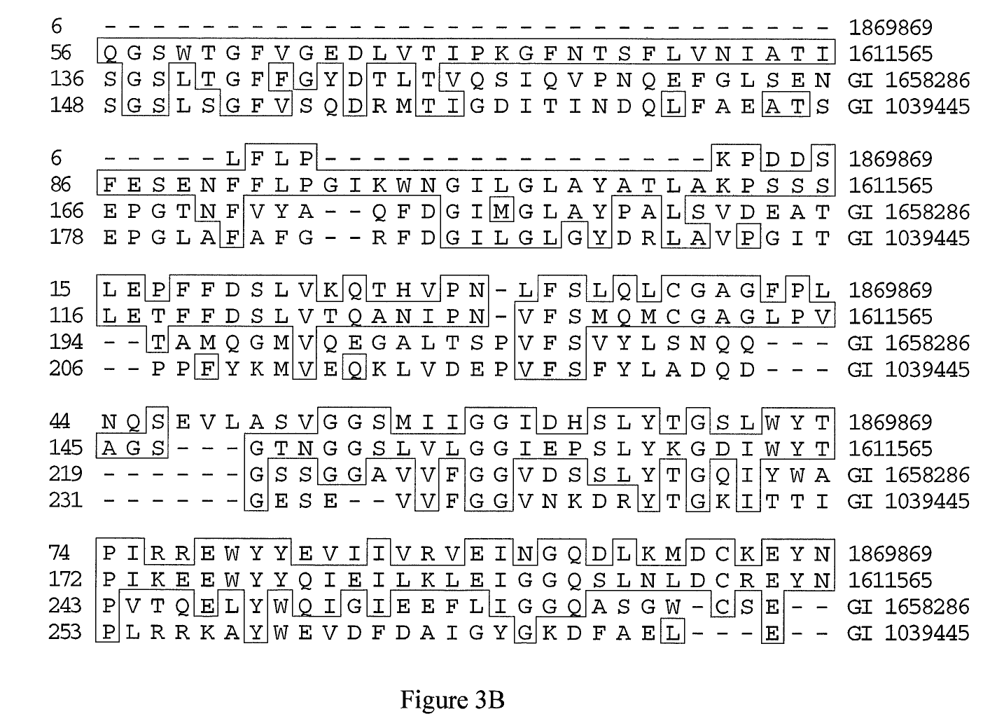

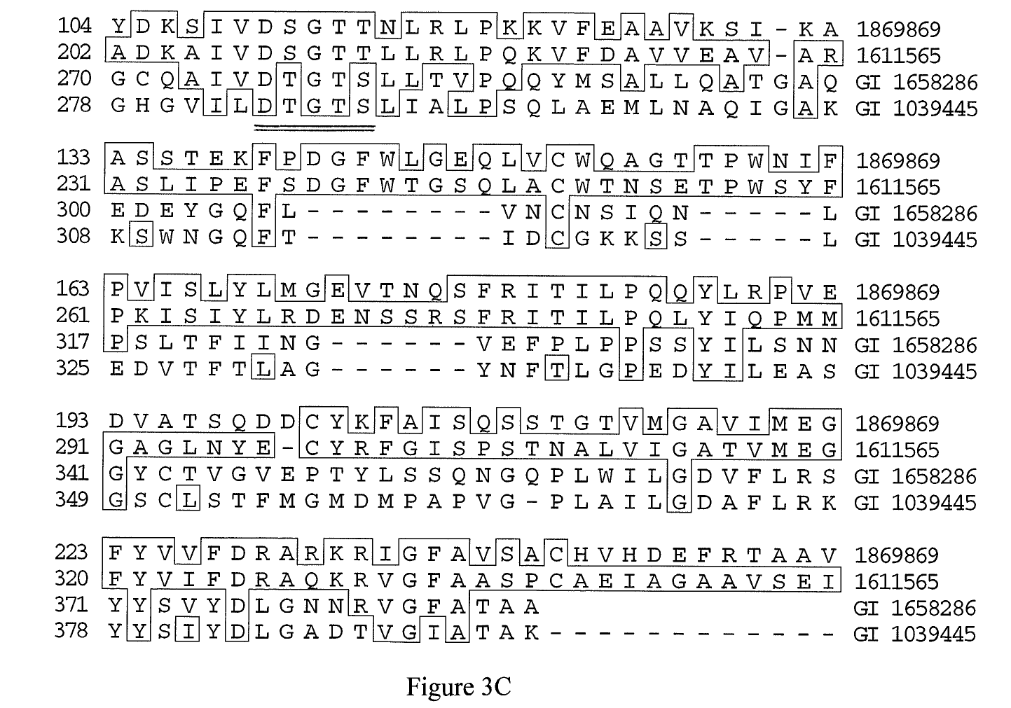

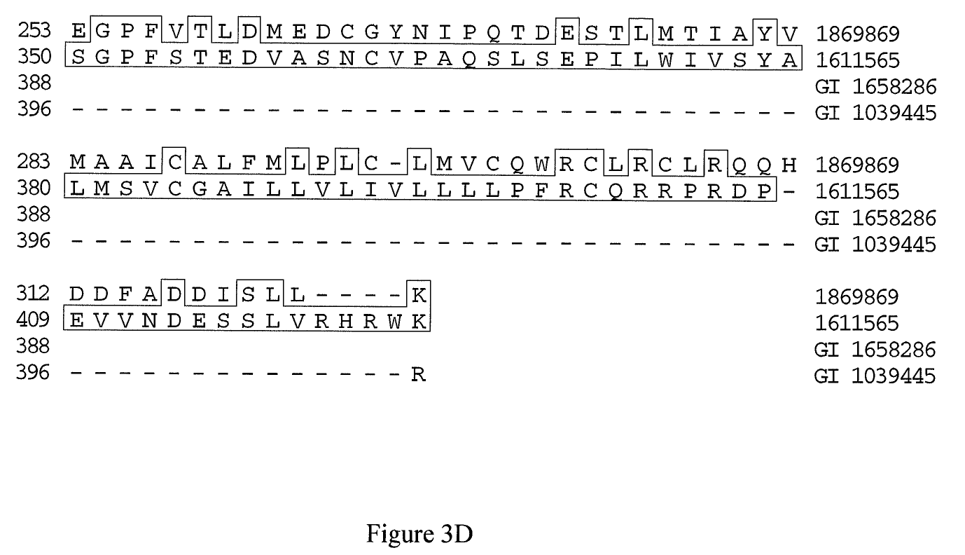

ASP comprises the amino acid sequences of SEQ ID NO:46, 423 amino acids in length, and of SEQ ID NO:47, 322 amino acids in length. The following chemical and structural characterization of the ASP1 (SEQ ID NO:46) will be based on absolute positions (30 residues per line) shown in FIGS. 3A, 3B, 3C, and 3D.

The human ASP1 protein (SEQ ID NO:46) has two potential N-glycosylation sites at N75 and N271; a leucine zipper site at residue K102; nine casein kinase II phosphorylation sites at residues A29, H33, L84, L114, S118, Q251, L315, I354, and C367; and five protein kinase C phosphorylation sites residues D37, T42, K51, L272, and E275. In addition, SEQ ID NO:46 has two eukaryotic and viral aspartyl protease sites at residues P12 and Y205. SEQ ID NO:46 shares 43.5% similarity with SEQ ID NO:47, 18.8% similarity with human gastricsin (SEQ ID NO:48; GI 1658286), and 14.1% similarity with Neurospora crassa vacuolar protease (SEQ ID NO:49; GI 1039445). Percent similarity was calculated using the MEGALIGN program of LASERGENE software (DNASTAR). The amino acids of SEQ ID NO:46 from residue T35 to residue G47 are useful for antibody production and were selected using the PROTEAN program of LASERGENE software (DNASTAR). BLOCKS analysis (p<10⁻¹²) confirmed the identity of SEQ ID NO:46 as an aspartyl protease based on amino acid sequence identity from L40 through G55, G129 through T137, G187 through G196, A235 through L244, V341 through A364 of SEQ. ID NO:46. In addition, PRINTS analysis (p<10⁻⁸) identifies two aspartyl protease signatures in SEQ ID NO:46 from residues I33 through V53 and G182 through K195, A235 through L244, V240 through D354 of SEQ ID NO:46.

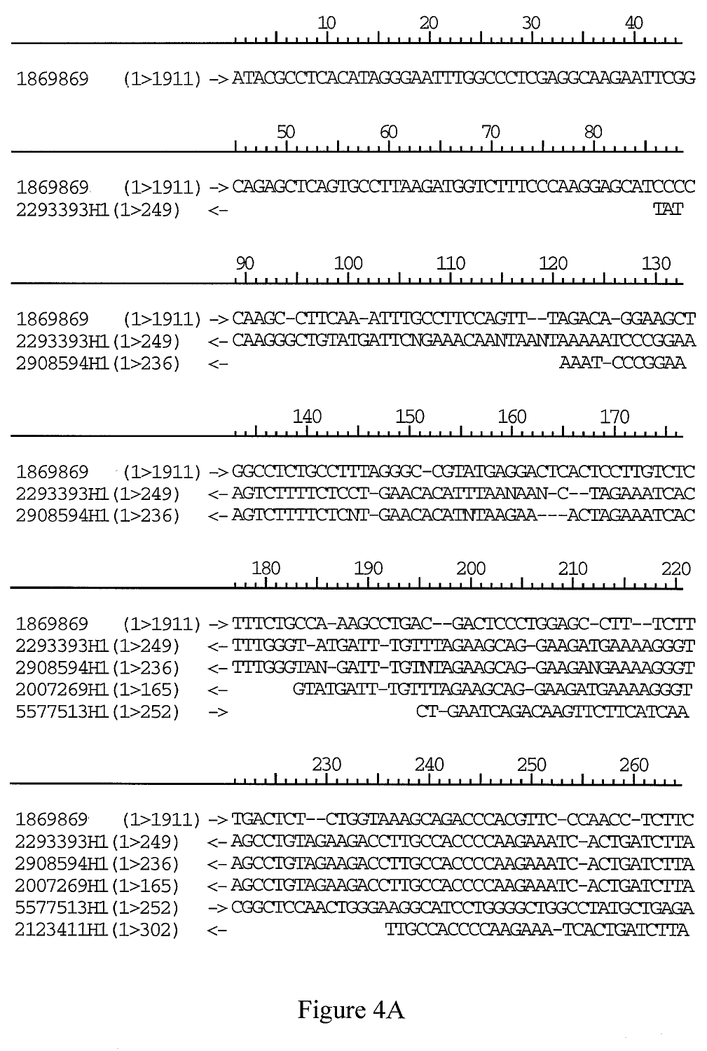

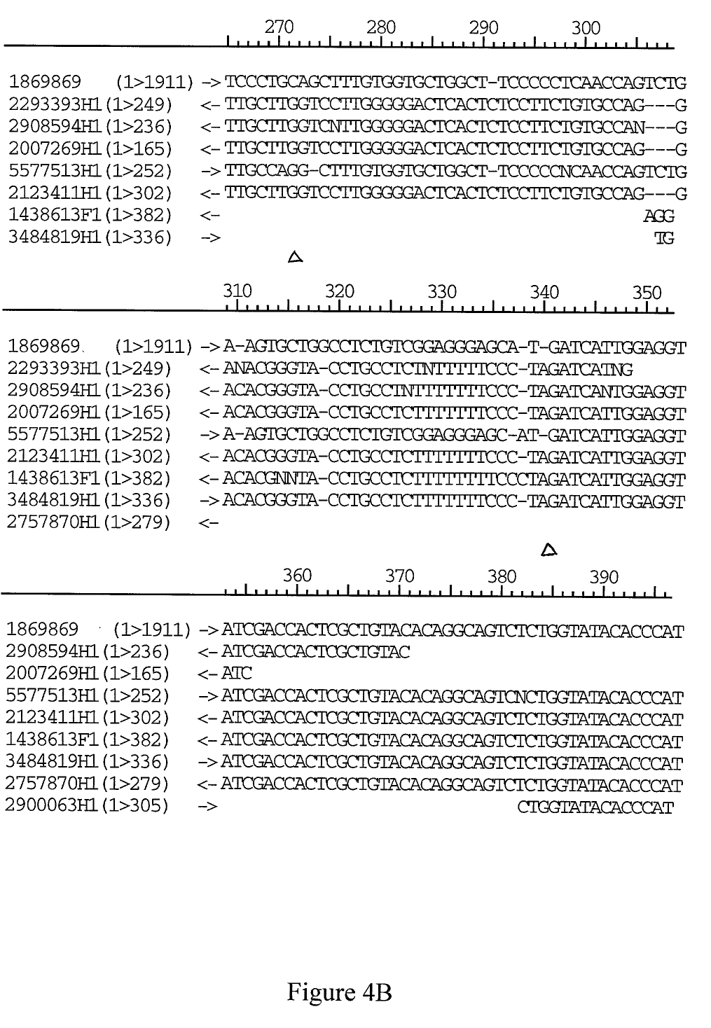

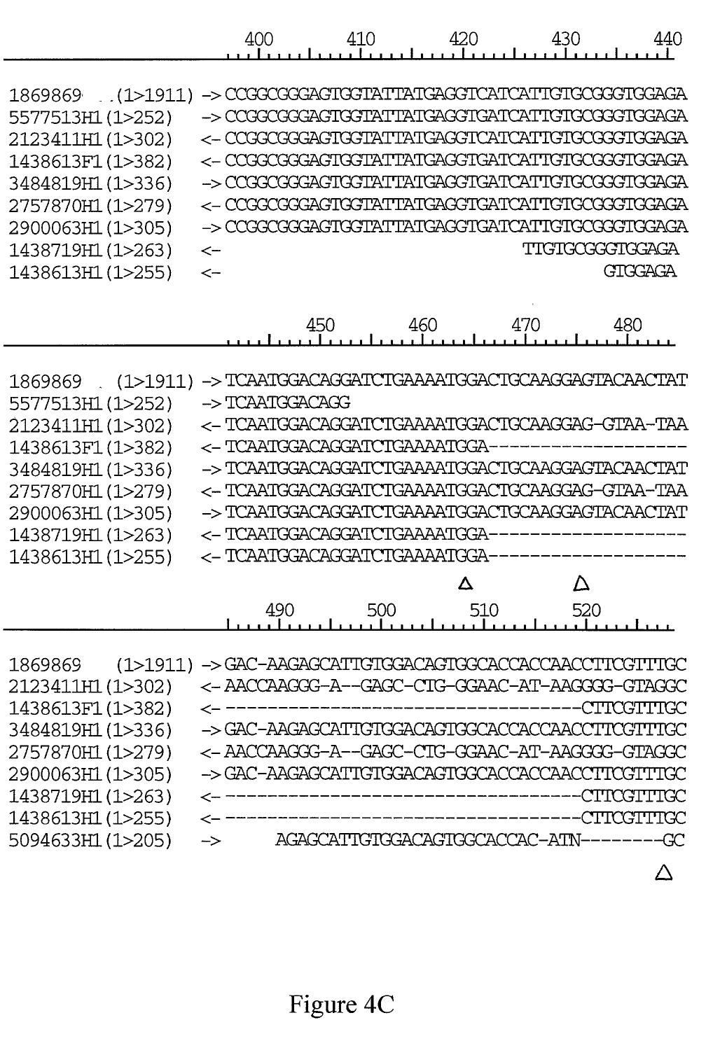

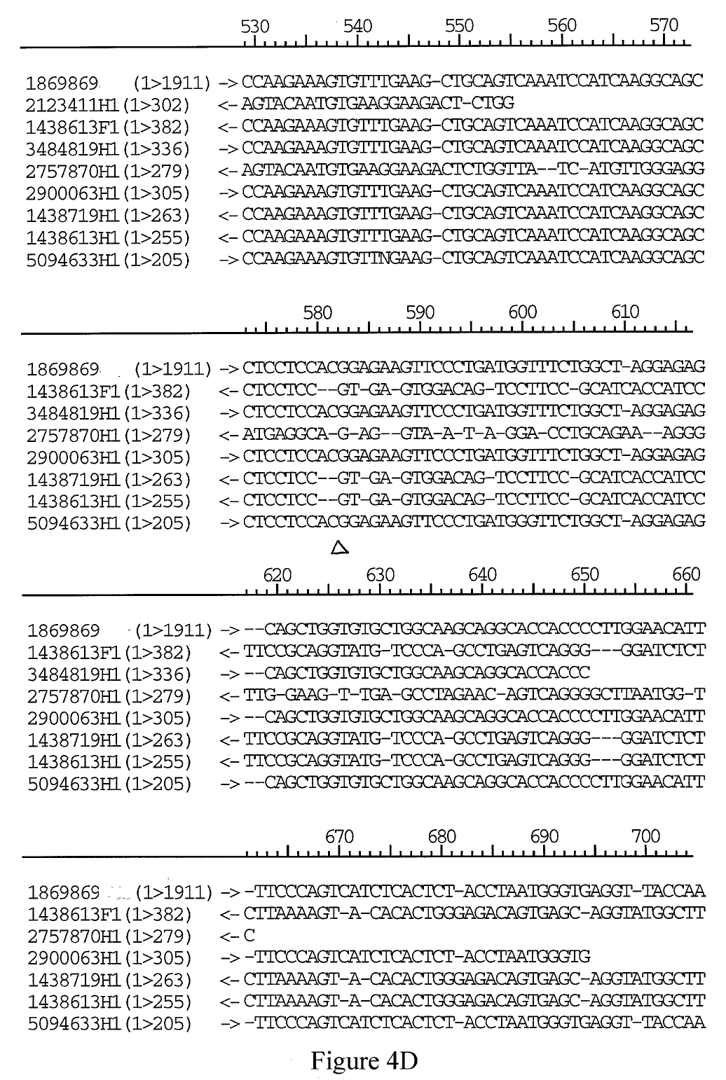

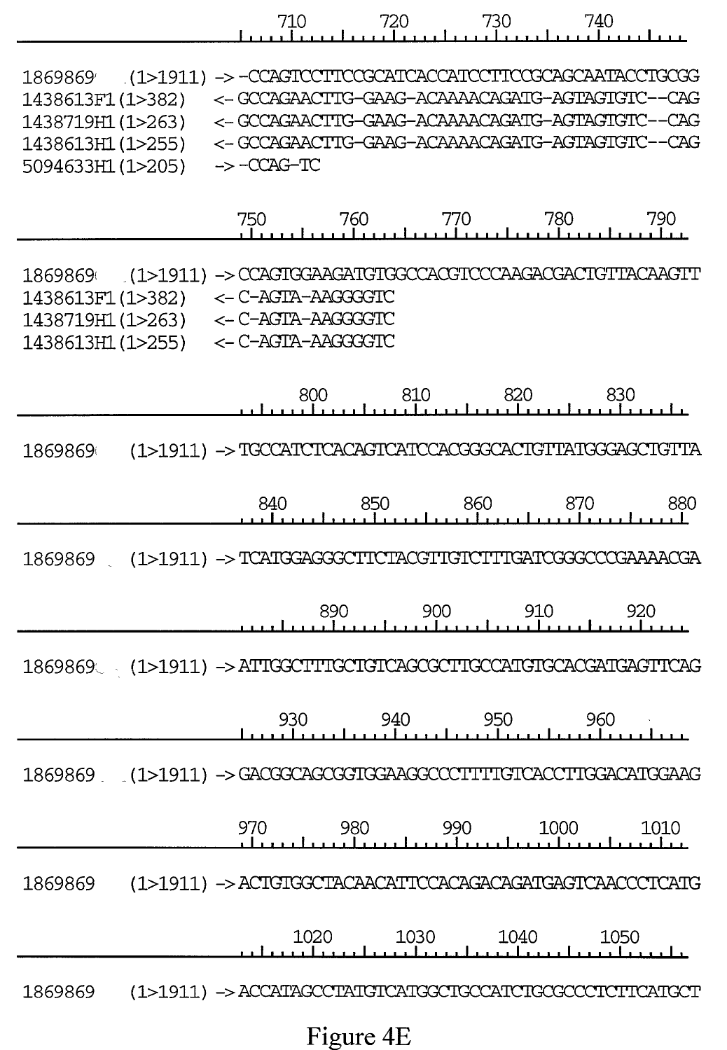

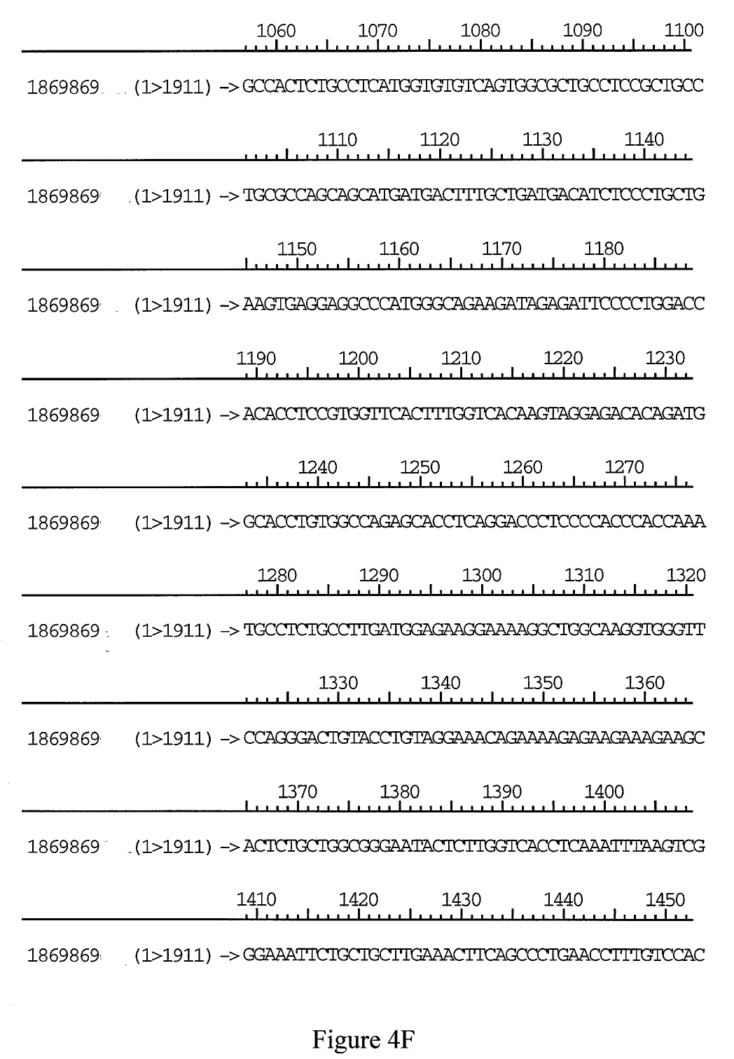

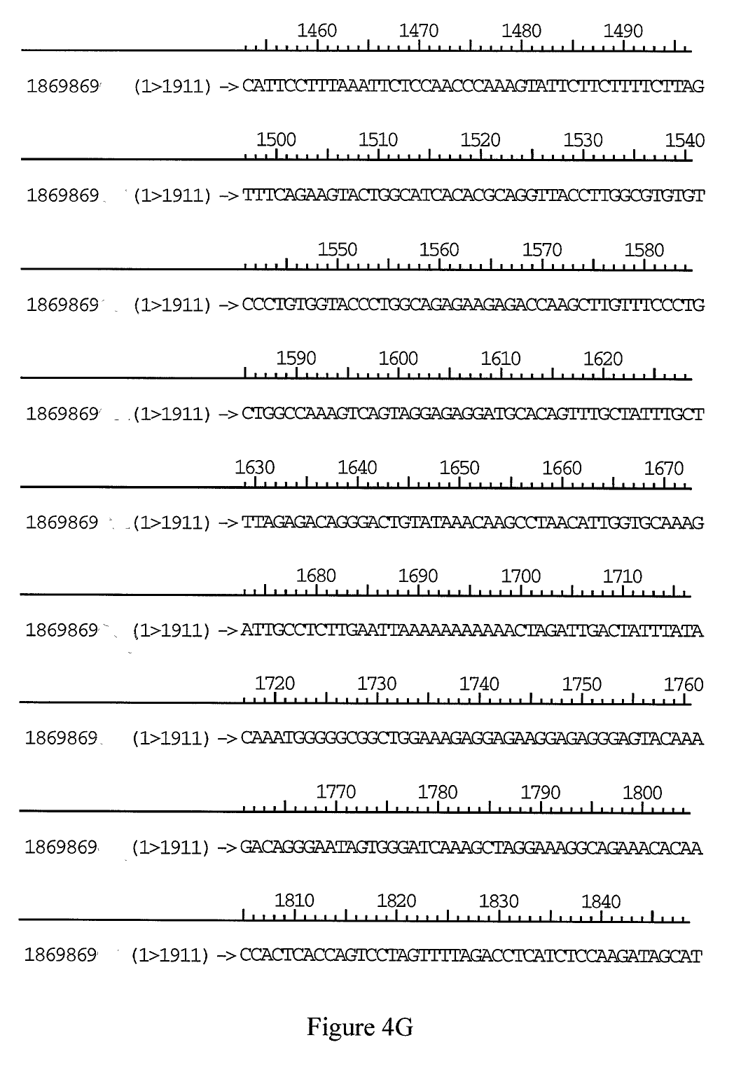

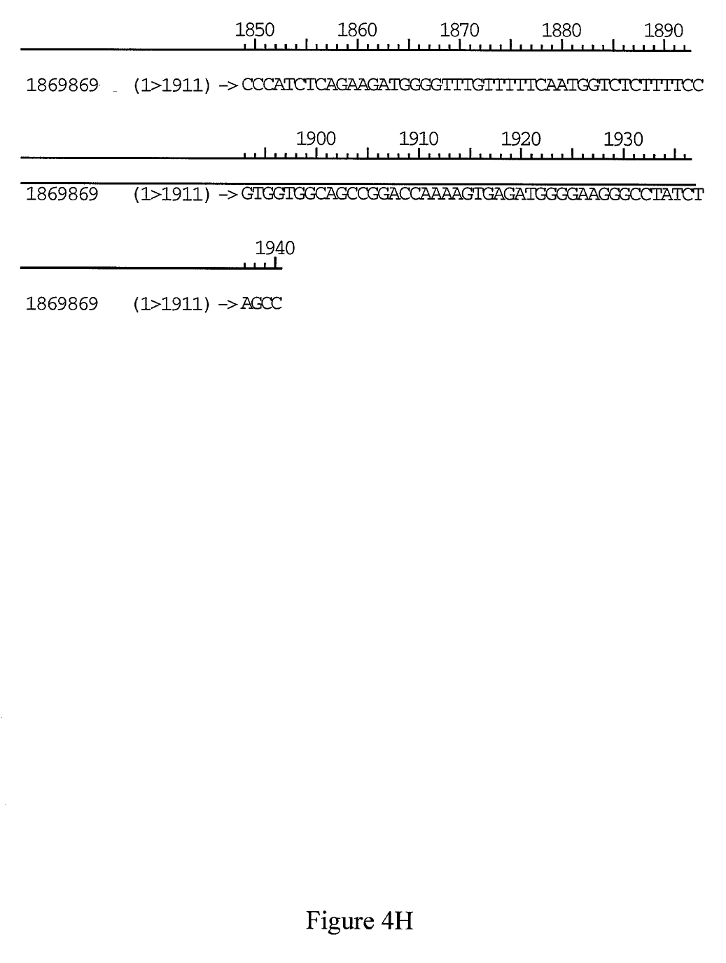

SEQ ID NO:44 was used to identify human ASP2 variant nucleic acid molecules in the Incyte LIFESEQ database (Incyte Pharmaceuticals). Incyte clones (libraries) 1499218H1 (SINTBST01), 1438719T6 (PANCNOT08), 3642853T6 (LUNGNOT34), 1499218F6 (SINTBST01), 1377178H1 and 1377178F1 (LUNGNOT10), 3642853F6 (LUNGNOT34), 5577513H1 (BRAPNOT04), 5094633H1 (EPIMNON05), 2293393H1 (BRAINON01), 2908594H1 (THYMNOT05), 2007269H1 (TESTNOT03), 1438613F1 (PANCNOT08), 3484819H1 (KIDNNOT31), 2757870H1 (THP1AZS08), 2900063H1 (DRGCNOT01), 2123411H1 (BRSTNOT07), 1438719H1 (PANCNOT08), and 1438613H1 (PANCNOT08), SEQ ID NOs: 17-28, respectively; are variants of the consensus sequence, SEQ ID NO:44 (FIGS. 4A, 4B, 4C, 4D, 4E, 4F, 4G, and 4H). Putative splice junction sites are indicated figures by (Δ). These clones were expressed in libraries with the following tissue, organ, or system associations: reproductive (3/15), cardiovascular (2/15), digestive system (4/15), neurological (3/15), hematopoietic (2/15), and urologic (1/15) tissues.

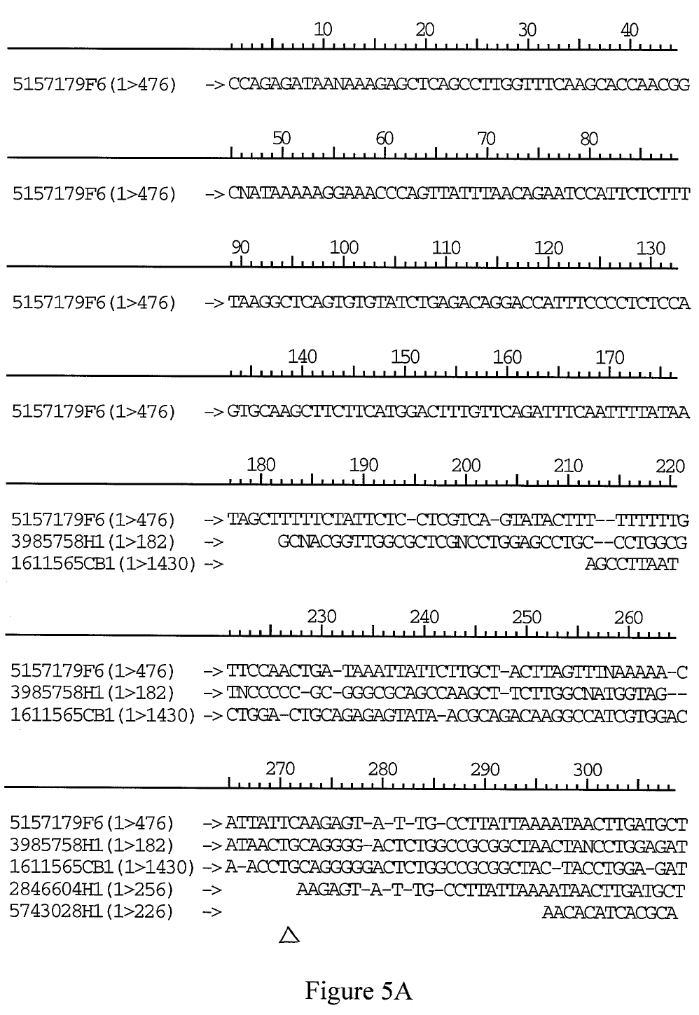

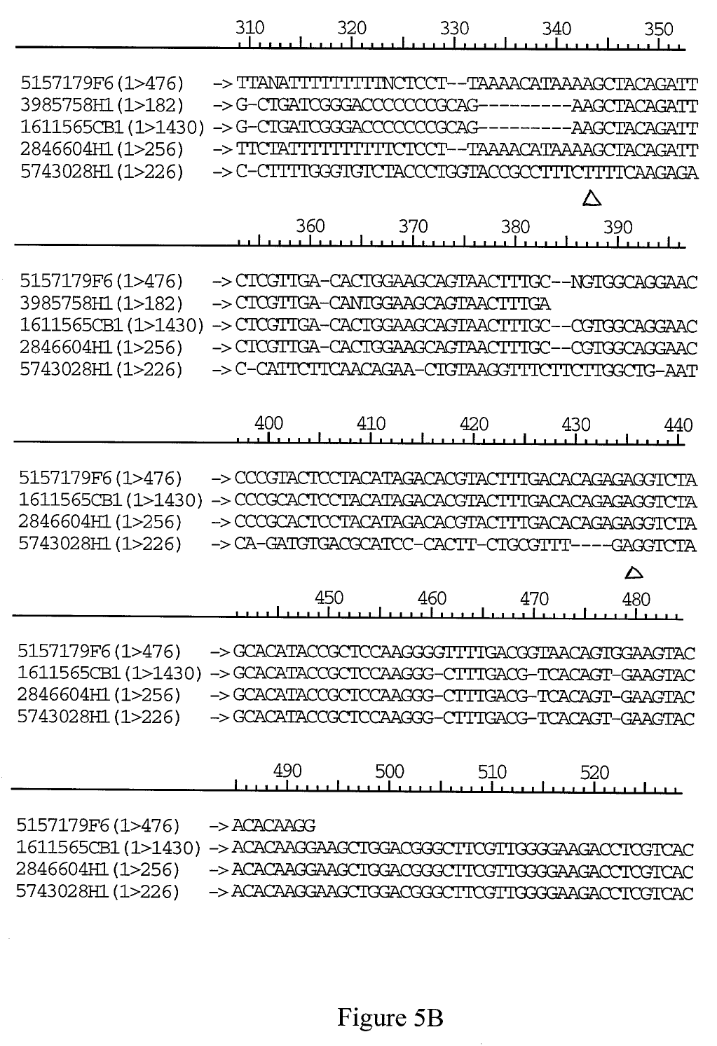

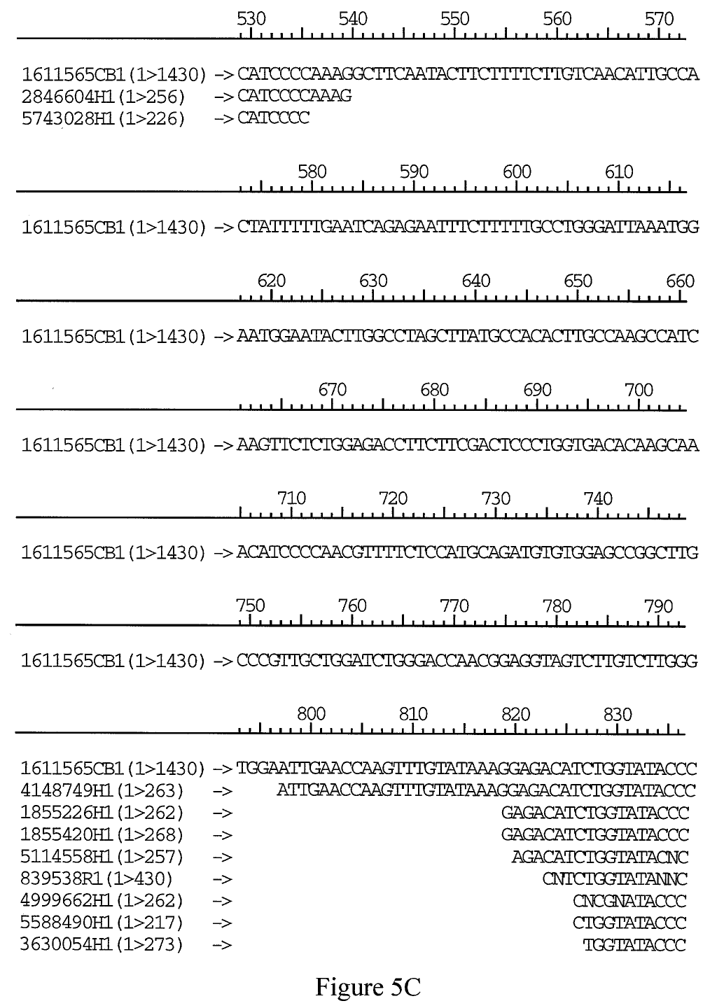

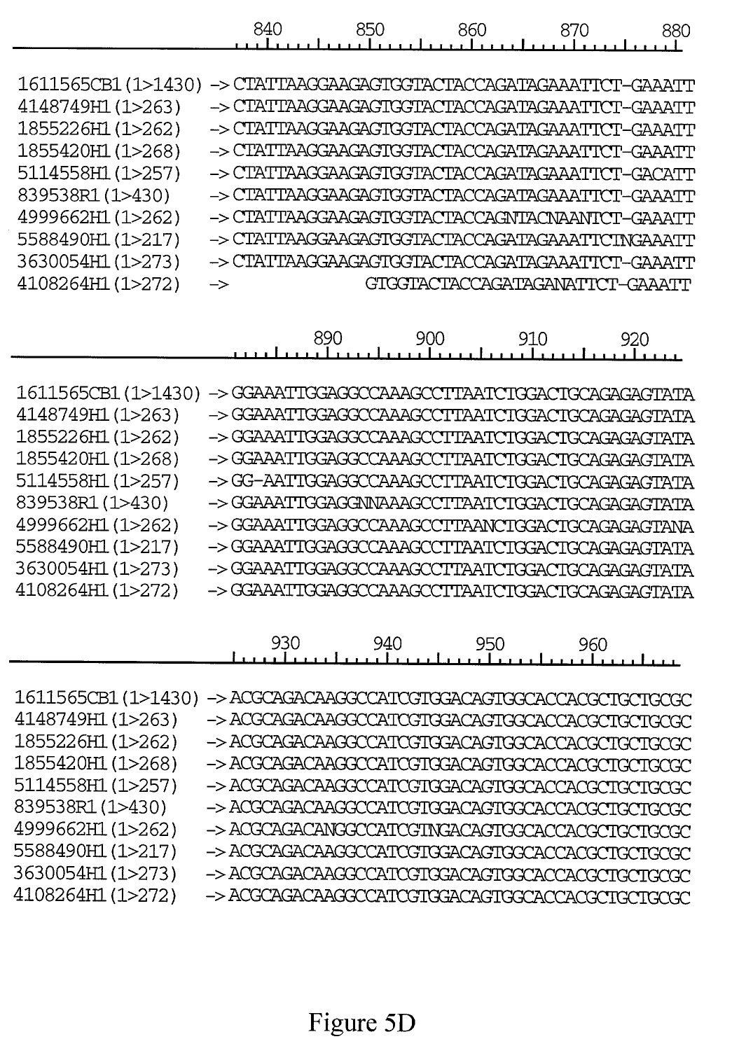

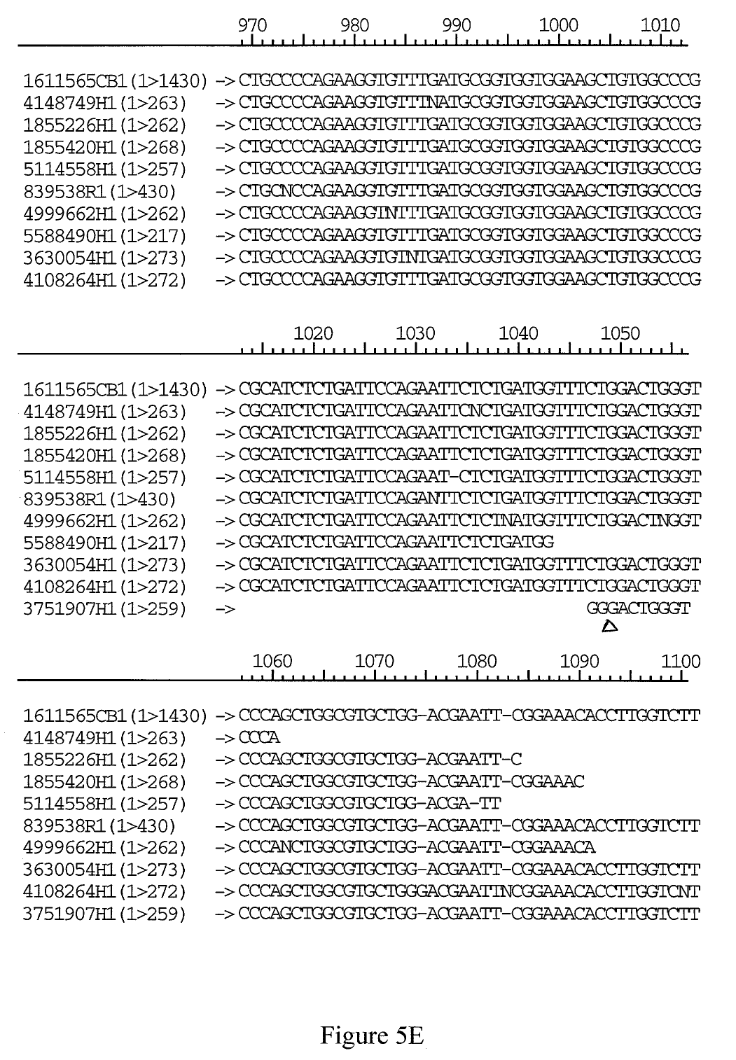

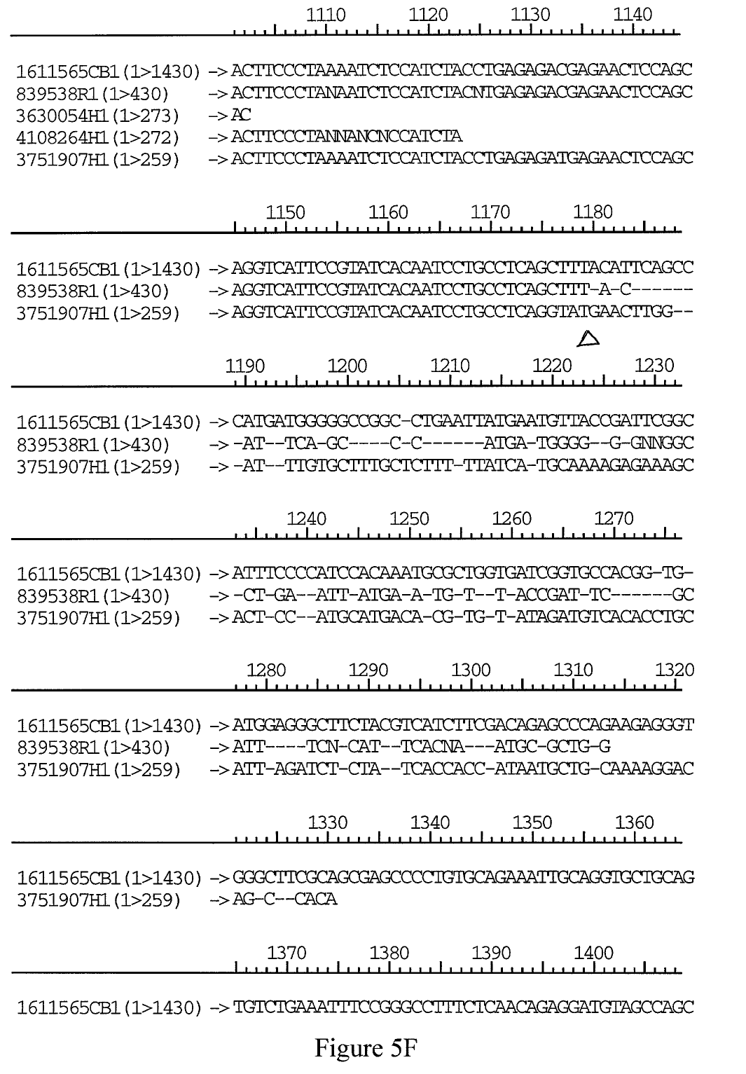

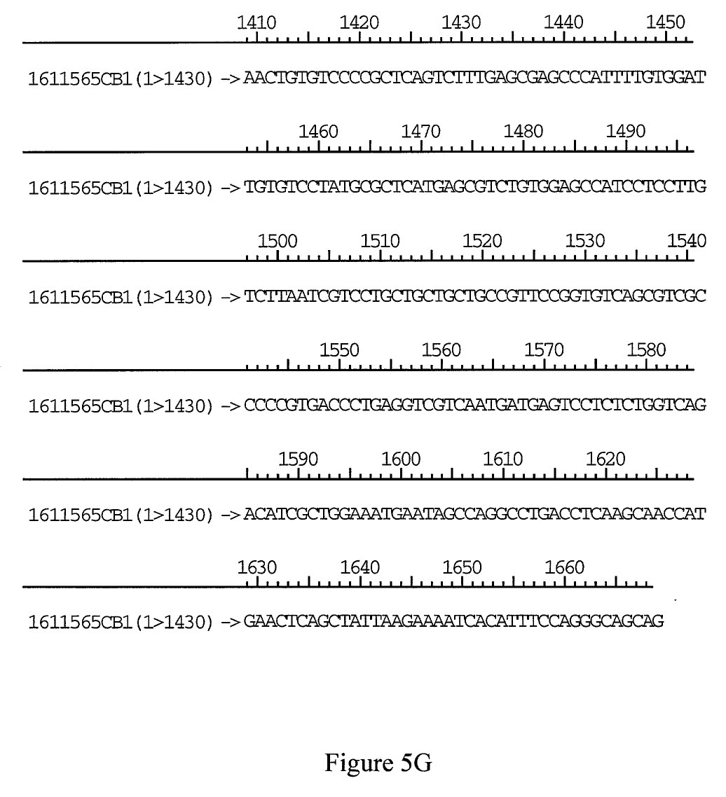

SEQ ID NO:43 was used to identify human ASP1 variant nucleic acid molecules in the Incyte LIFESEQ database (Incyte Pharmaceuticals). Incyte clones (libraries) 5157179F6 (BRSTTMT02), 3985758H1 (UTRSTUT05), 2846604H1 (DRGLNOT01), 5743028H1 (LUNGNON03), 3751907H1 (UTRSNOT18), 4148749H1 (SINITUT04), 1855226H1 and 1855420H1 (HNT3AZT01), 5114558H1 (ENDITXT01), 839538R1 (PROSTUT05), 5588490H1 (ENDINOT02), 4999662H1 (MYEPTXT02), 3630054H1 (COLNNOT38), and 4108264H1 (PROSBPT07), SEQ ID NOs:29-42, respectively; are variants of the consensus sequence, SEQ ID NO:43 (FIGS. 5A, 5B, 5C, 5D, 5E, 5F and 5G). Putative splice junction sites are indicated on the figures by (Δ). These clones were expressed in libraries with the following cell, tissue, organ, or system associations: reproductive (5/15), cardiovascular (4/15), digestive system (2/15), thoracic ganglion (1/15), hematopoietic (2/15) tissues, and teratocarcinoma cell line (2/15).

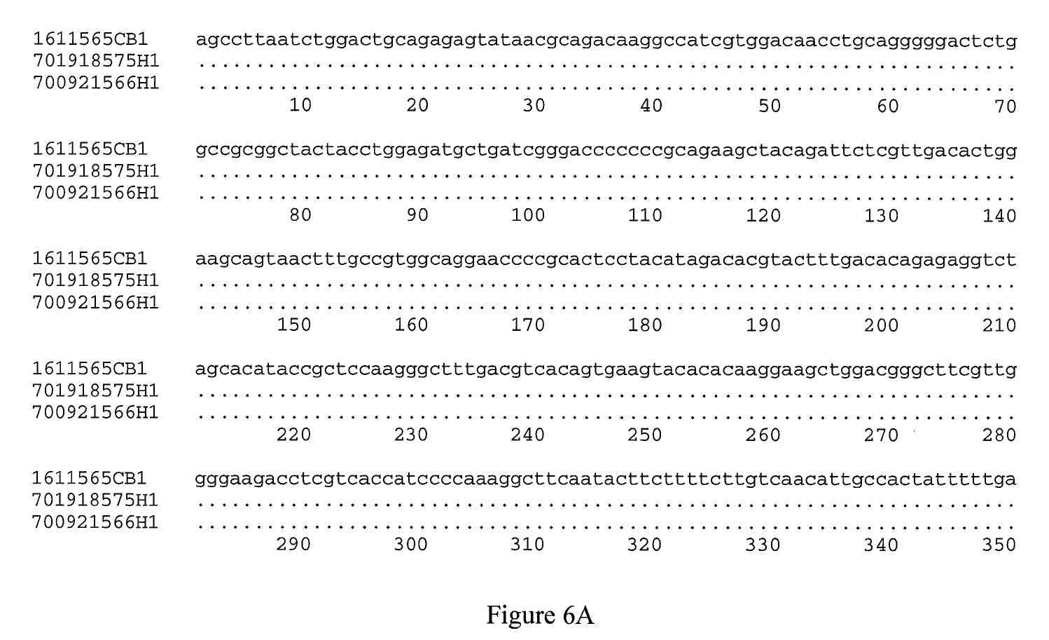

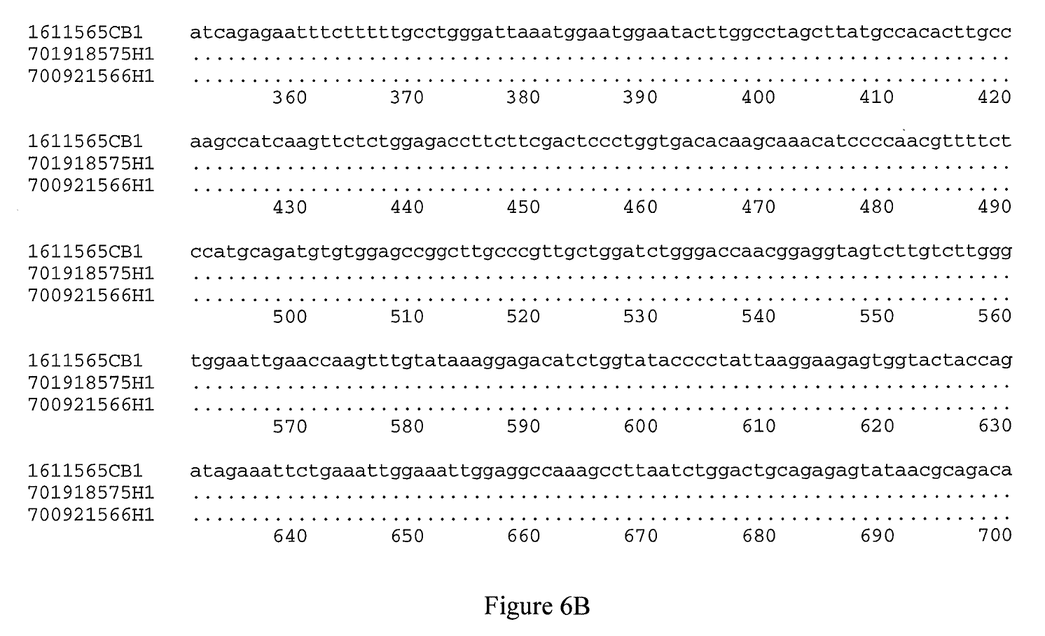

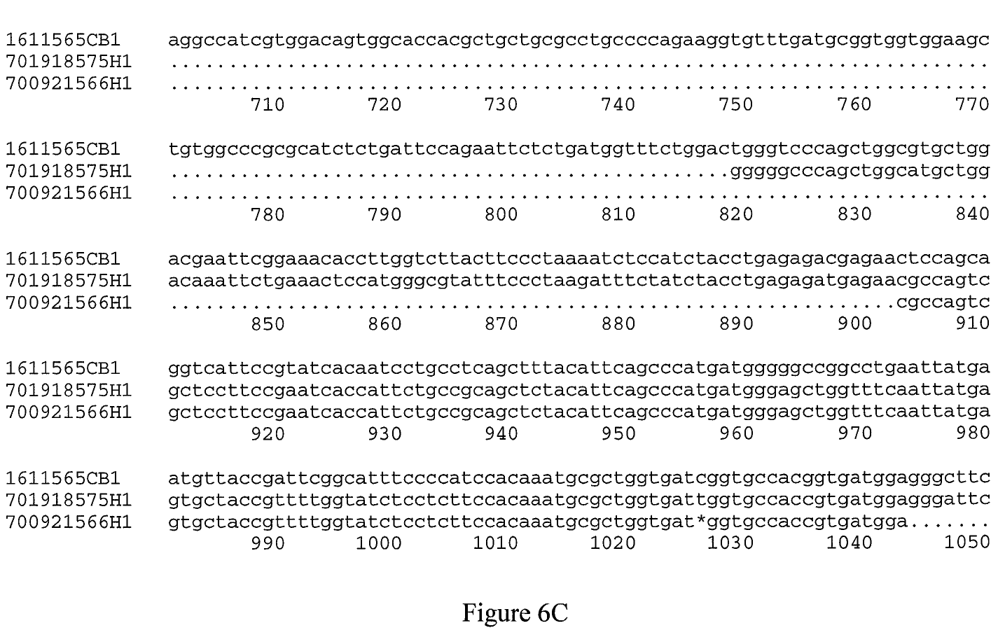

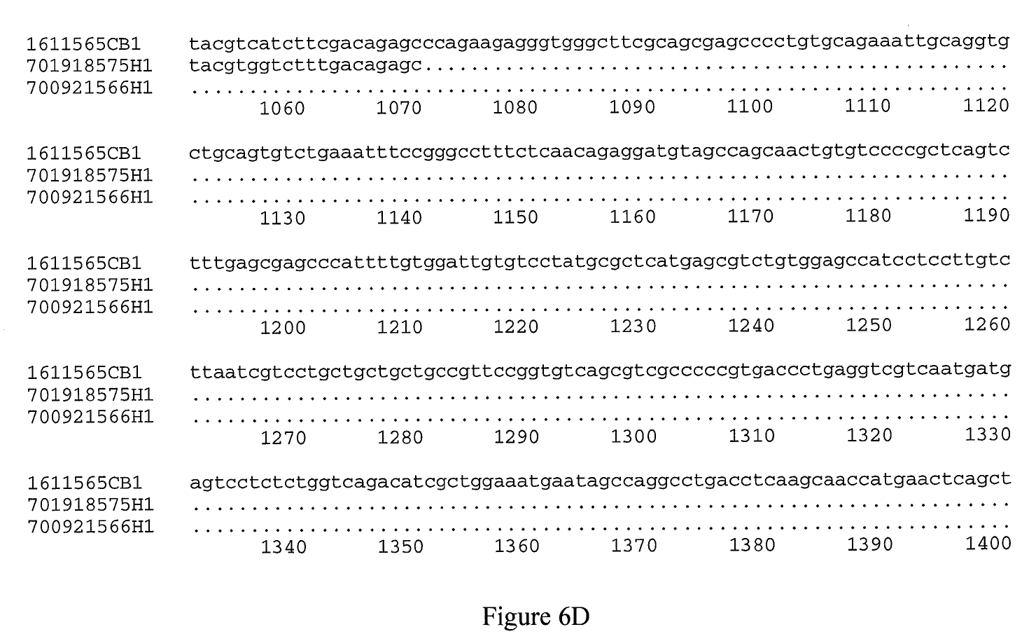

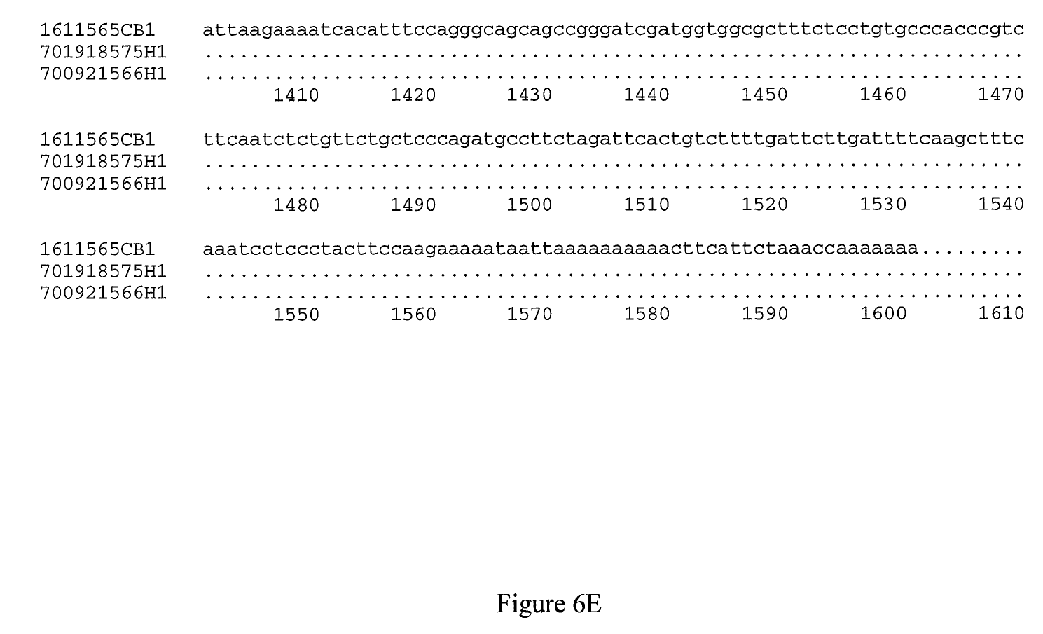

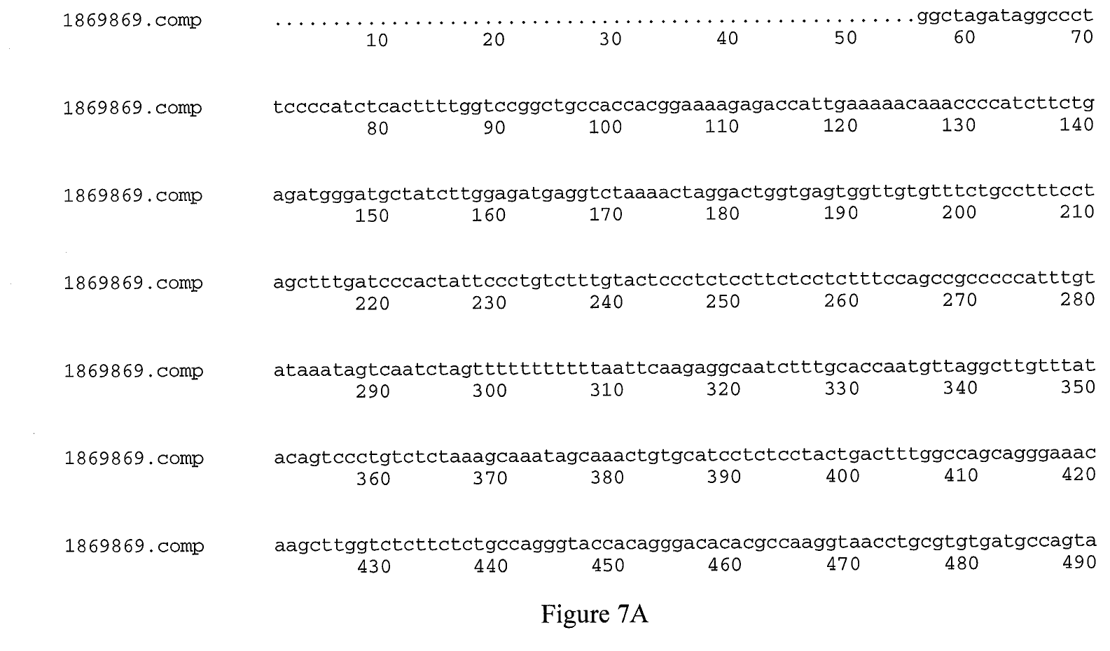

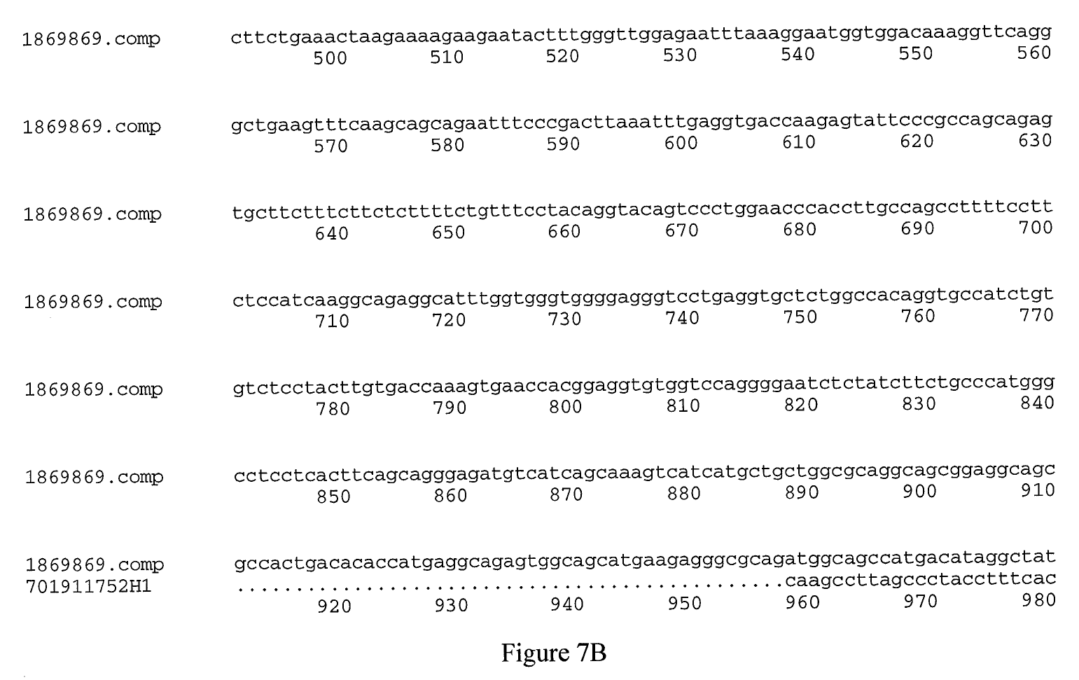

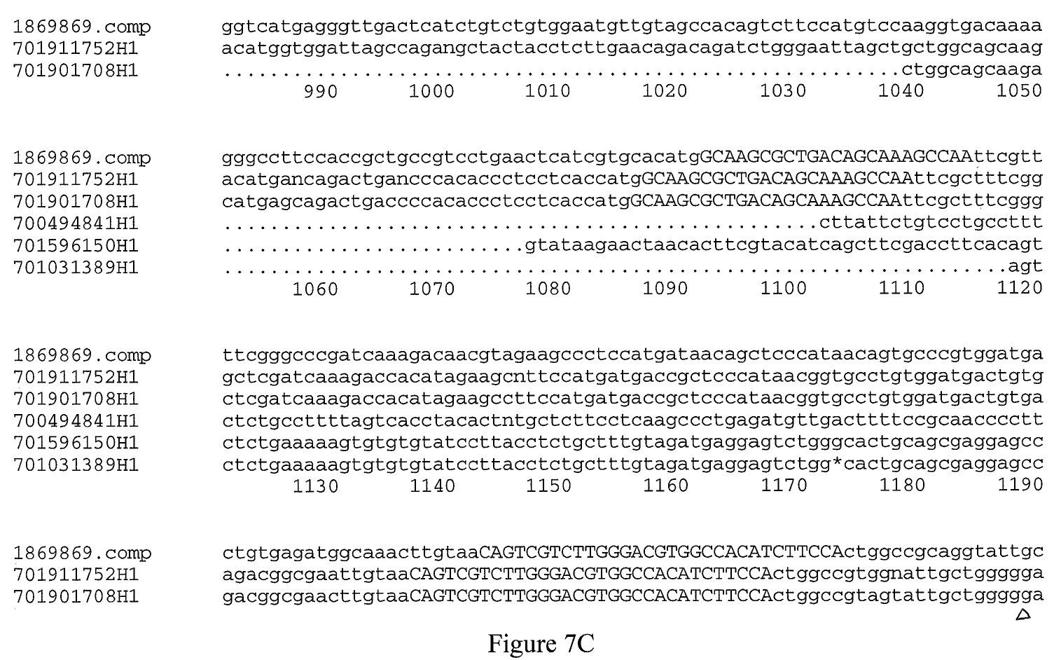

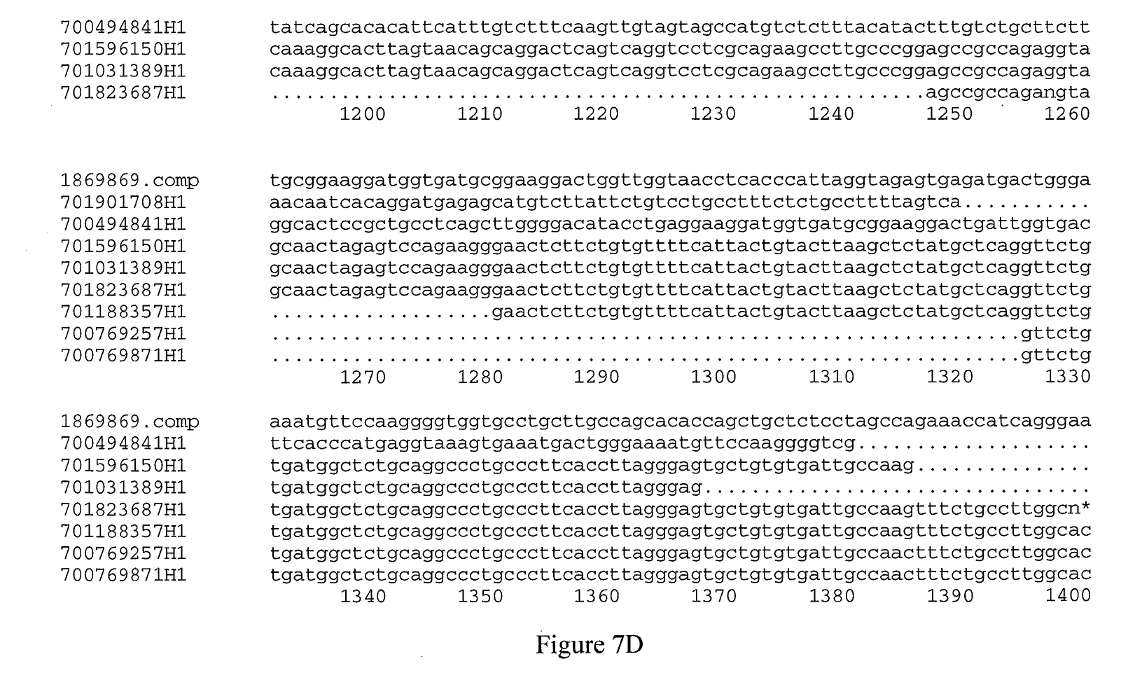

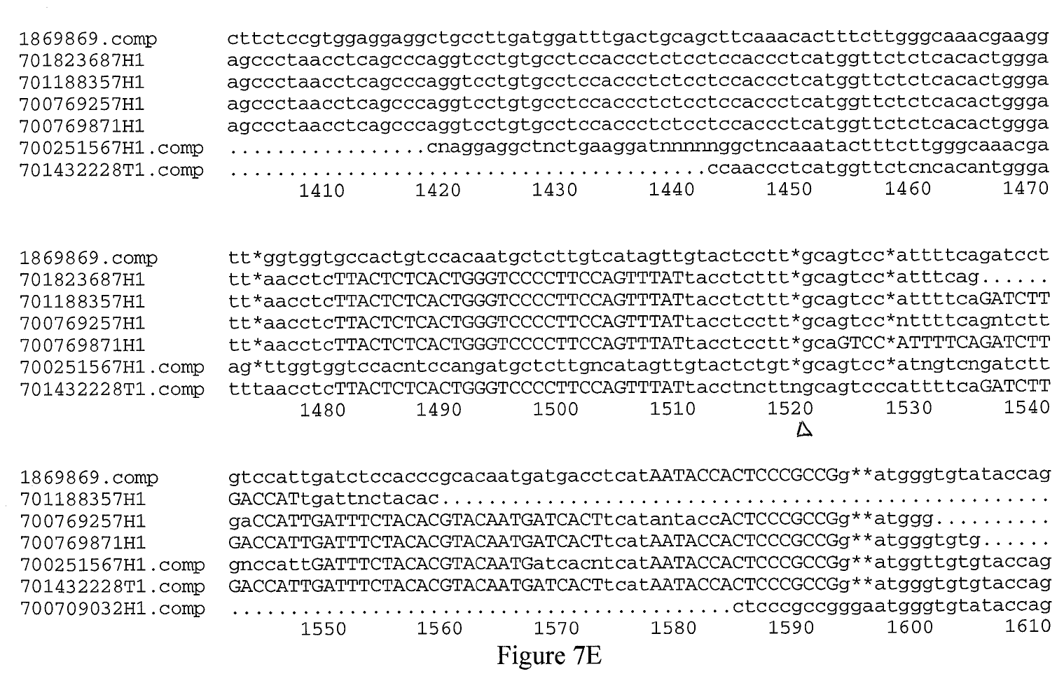

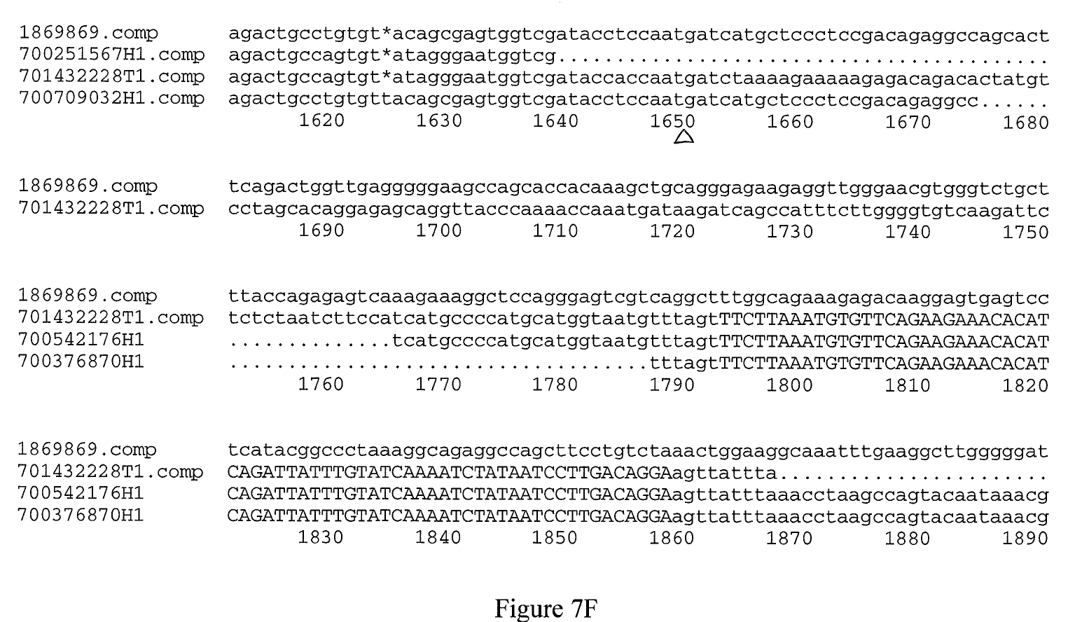

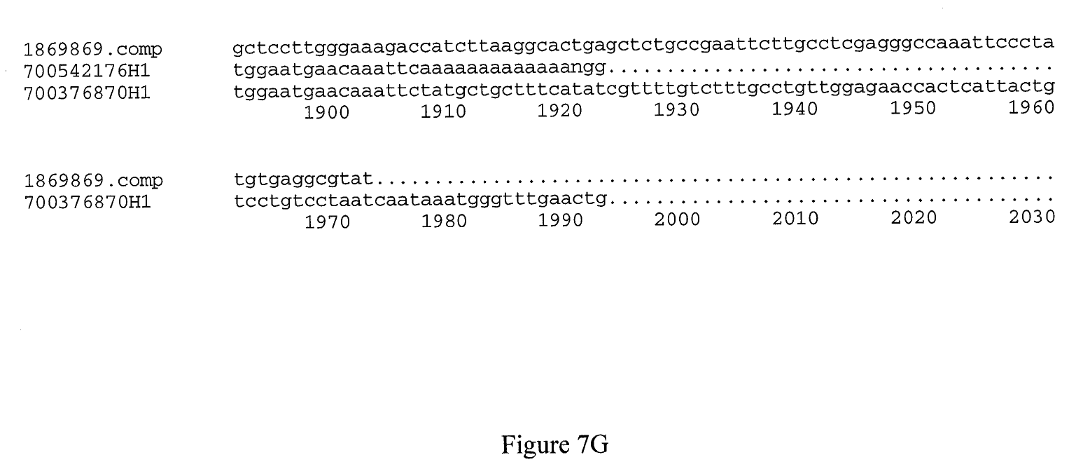

Additional nucleic acid molecules encoding the mammalian aspartyl protease of the present invention were identified by using BLAST or BLAST2 (Altschul et al. (1997) Nucleic Acids Res. 25:3389-3402) against the ZOOSEQ database (Incyte Pharmaceuticals, Palo Alto Calif.) to align rat and monkey clones with the human aspartyl protease (SEQ ID NOs:43 and 44). The clones (from various libraries): 700251567H1 (RABMNOT01), 701432228T1 (RALITXT06), 700769871H1 (RAHYNOT02), 700769257H1 (RAHYNOT02), 701188357H1 (RACONON02), 701918575H1 (RABYUNS03), 700921566H1 (RANPNOT02), 700494841H1 (RABCNOT01), 700885432H1 (RAVANOT01), 701901708H1 (RABYUNS09), 701911752H1 (RABYUNS03), 701823687H1 (RAKITXT08), 701596150H1 (RALITXT26), 700542176H1 (RACONOT01), 701031389H1 (RABMNON02), 700376870H1 (RALANOT01), and 700709032H1 (MNBFNOT01) presented in the Sequence Listing as SEQ ID NOs: 1-16, respectively; are non-human variants of the nucleic acid molecules, SEQ ID NOs:43 and 44, which encode ASPs (FIGS. 6A, 6B, 6C, 6D, 6E and FIGS. 7A, 7B, 7C, 7D, 7E, 7G, respectively). Putative splice junction sites are indicated on the figures by (Δ). These clones were expressed in libraries with the following tissue, organ, or system associations: neurological (10/16), digestive system (4/16), cardiovascular (1/16), and urologic (1/16) tissues.

The nucleic acid sequences, SEQ ID NOs: 17-42 may be used in hybridization and amplification technologies to identify and distinguish among SEQ ID NOs:43 and 44 and similar molecules in a sample. The molecules may be used to mimic human conditions, diseases, or disorders, produce transgenic animal models for these conditions, or to monitor animal toxicology studies, clinical trials, and subject/patient treatment profiles.

### Characterization and Use of the Invention

### cDNA Libraries

In a particular embodiment disclosed herein, mRNA was isolated from mammalian cells and tissues using methods that are well known to those skilled in the art and used to prepare the cDNA libraries. The Incyte clones listed above were isolated from mammalian cDNA libraries. At least one library preparation representative of the invention is described in the EXAMPLES below. The consensus mammalian sequences were chemically and/or electronically assembled from fragments including Incyte clones and extension and/or shotgun sequences using computer programs such as Phrap (Green, supra), GELVIEW Fragment Assembly system (Genetics Computer Group, Madison Wis.), and AUTOASSEMBLER application (PE Biosystems, Foster City Calif.). Clones, extension and/or shotgun sequences are electronically assembled into clusters and/or master clusters.

### Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the HYDRA microdispenser (Robbins Scientific, Sunnyvale Calif.), MICROLAB 2200 system (Hamilton, Reno Nev.), and the DNA ENGINE thermal cycler (PTC200; MJ Research, Watertown Mass.). Machines used for sequencing include the ABI PRISM 3700, 377 or 373 DNA sequencing systems (PE Biosystems), the MEGABACE 1000 DNA sequencing system (Amersham Pharmacia Biotech), and the like. The sequences may be analyzed using a variety of algorithms which are well known in the art and described in Ausubel et al. (1997; Short Protocols in Molecular Biology, John Wiley & Sons, New York N.Y., unit 7.7) and Meyers (1995; Molecular Biology and Biotechnology, Wiley VCH, New York N.Y., pp. 856-853).

Shotgun sequencing is used to generate more sequence from cloned inserts derived from multiple sources. Shotgun sequencing methods are well known in the art and use thermostable DNA polymerases, heat-labile DNA polymerases, and primers chosen from representative regions flanking the nucleic acid molecules of interest. Incomplete assembled sequences are inspected for identity using various algorithms or programs such as CONSED (Gordon (1998) Genome Res. 8:195-202) which are well known in the art. Contaminating sequences including vector or chimeric sequences or deleted sequences can be removed or restored, respectively, organizing the incomplete assembled sequences into finished sequences.

### Extension of a Nucleic Acid Sequence

The sequences of the invention may be extended using various PCR-based methods known in the art. For example, the XL-PCR kit (PE Biosystems), nested primers, and commercially available cDNA or genomic DNA libraries may be used to extend the nucleic acid sequence. For all PCR-based methods, primers may be designed using commercially available software, such as OLIGO 4.06 primer analysis software (National Biosciences, Plymouth Minn.) to be about 22 to 30 nucleotides in length, to have a GC content of about 50% or more, and to anneal to a target molecule at temperatures from about 55° C. to about 68° C. When extending a sequence to recover regulatory elements, it is preferable to use genomic, rather than cDNA libraries.

### Use of the Mammalian Nucleic Acid Molecule

### Hybridization

The mammalian nucleic acid molecule and fragments thereof can be used in hybridization technologies for various purposes. A probe may be designed or derived from unique regions such as the 5′ regulatory region or from a conserved motif such as the aspartyl protease signature and used in protocols to identify naturally occurring molecules encoding the mammalian protein, allelic variants, or related molecules. The probe may be DNA or RNA, is usually single stranded and should have at least 50% sequence identity to any of the nucleic acid sequences. The probe may comprise at least 16 contiguous nucleotides of a nucleic acid molecule. Hybridization probes may be produced using oligolabeling, nick translation, end-labeling, or PCR amplification in the presence of labeled nucleotide. A vector containing the nucleic acid molecule or a fragment thereof may be used to produce an mRNA probe in vitro by addition of an RNA polymerase and labeled nucleotides. These procedures may be conducted using commercially available kits such as those provided by Amersham Pharmacia Biotech.

The stringency of hybridization is determined by G+C content of the probe, salt concentration, and temperature. In particular, stringency can be increased by reducing the concentration of salt or raising the hybridization temperature. In solutions used for some membrane based hybridizations, addition of an organic solvent such as formamide allows the reaction to occur at a lower temperature. Hybridization can be performed at low stringency with buffers, such as 5×SSC with 1% sodium dodecyl sulfate (SDS) at 60° C., which permits the formation of a hybridization complex between nucleic acid sequences that contain some mismatches. Subsequent washes are performed at higher stringency with buffers such as 0.2×SSC with 0.1% SDS at either 45° C. (medium stringency) or 68° C. (high stringency). At high stringency, hybridization complexes will remain stable only where the nucleic acid molecules are completely complementary. In some membrane-based hybridizations, preferably 35% or most preferably 50%, formamide can be added to the hybridization solution to reduce the temperature at which hybridization is performed, and background signals can be reduced by the use of other detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Microarrays may be prepared and analyzed using methods known in the art. Oligonucleotides may be used as either probes or targets in a microarray. The microarray can be used to monitor the expression level of large numbers of genes simultaneously and to identify genetic variants, mutations, and single nucleotide polymorphisms. Such information may be used to determine gene function; to understand the genetic basis of a condition, disease, or disorder; to diagnose a condition, disease, or disorder; and to develop and monitor the activities of therapeutic agents. (See, e.g., Brennan et al. (1995) USPN 5,474,796; Schena et al. (1996) Proc. Natl. Acad. Sci. 93:10614-10619; Baldeschweiler et al. (1995) PCT application WO95/251116; Shalon et al. (1995) PCT application WO95/35505; Heller et al. (1997) Proc. Natl. Acad. Sci. 94:2150-2155; and Heller et al. (1997) U.S. Pat. No. 5,605,662.)

Hybridization probes are also useful in mapping the naturally occurring genomic sequence. The probes may be hybridized to: 1) a particular chromosome, 2) a specific region of a chromosome, 3) artificial chromosome constructions such as human artificial chromosomes (HAC), yeast artificial chromosomes (YAC), bacterial artificial chromosomes (BAC), bacterial P1 constructions, or single chromosomes, or cDNA libraries made from any of these sources.

### Expression

A multitude of nucleic acid molecules encoding ASP may be cloned into a vector and used to express the protein, or portions thereof, in host cells. The nucleic acid sequence can be engineered by such methods as DNA shuffling (Stemmer and Crameri (1996) U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources that have been selected for their efficiency in a particular host. The vector, nucleic acid molecule, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements, or animal cell systems (Ausubel supra, unit 16). For example, an adenovirus transcription/translation complex may be utilized in mammalian cells. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells that successfully express the introduced sequences. Resistant clones identified either by survival on selective media or by the expression of visible markers, such as anthocyanins, green fluorescent protein (GFP), B glucuronidase, luciferase, and the like, may be propagated using culture techniques. Visible markers are also used to quantify the amount of protein expressed by the introduced genes. Verification that the host cell contains the desired mammalian nucleic acid molecule is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing that cleaves a “prepro” form may also be used to specify protein targeting, folding, and/or activity. Different host cells available from the ATCC (Manassas Md.) which have specific cellular machinery and characteristic mechanisms for post-translational activities may be chosen to ensure the correct modification and processing of the recombinant protein.

### Recovery of Proteins from Cell Culture

Heterologous moieties engineered into a vector for ease of purification include glutathione S-transferase (GST), calmodulin binding peptide (CBP), 6-His, FLAG, MYC, and the like. GST, CBP, and 6-His are purified using commercially available affinity matrices such as immobilized glutathione, calmodulin, and metal-chelate resins, respectively. FLAG and MYC are purified using commercially available monoclonal and polyclonal antibodies. A proteolytic cleavage site may be located between the desired protein sequence and the heterologous moiety for ease of separation following purification. Methods for recombinant protein expression and purification are discussed in Ausubel (supra, unit 16) and are commercially available.

### Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the ABI 431 A peptide synthesizer (PE Biosystems). A protein or portion thereof may be substantially purified by preparative high performance liquid chromatography and its composition confirmed by amino acid analysis or by sequencing (Creighton (1984) Proteins, Structures and Molecular Properties, WH Freeman, New York N.Y.).

### Preparation and Screening of Antibodies

Various hosts including goats, rabbits, rats, mice, humans, and others may be immunized by injection with ASP or any portion thereof. Adjuvants such as Freund's, mineral gels, and surface active substances such as lysolecithin, pluronic polyols, polyanions, peptides, oil emulsions, keyhole limpet hemacyanin (KLH), and dinitrophenol may be used to increase immunological response. The oligopeptide, peptide, or portion of protein used to induce antibodies should consist of at least about five amino acids, more preferably ten amino acids, which are identical to a portion of the natural protein. Oligopeptides may be fused with proteins such as KLH in order to produce the chimeric molecule.

Monoclonal antibodies may be prepared using any technique that provides for the production of antibodies by continuous cell lines in culture. These include, but are not limited to, the hybridoma technique, the human B-cell hybridoma technique, and the EBV-hybridoma technique. (See, e.g., Kohler et al. (1975) Nature 256:495-497; Kozbor et al. (1985) J. Immunol. Methods 81:31-42; Cote et al. (1983) Proc. Natl. Acad. Sci. 80:2026-2030; and Cole et al. (1984) Mol. Cell. Biol. 62:109-120.)

Alternatively, techniques described for the production of single chain antibodies may be adapted, using methods known in the art, to produce epitope specific single chain antibodies. Antibody fragments which contain specific binding sites for epitopes of the mammalian protein may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

The mammalian protein or a portion thereof may be used in screening assays of phagemid or B-lymphocyte immunoglobulin libraries to identify antibodies having the desired specificity. Numerous protocols for competitive binding or immunoassays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering epitopes is preferred, but a competitive binding assay may also be employed (Pound (1998) Immunochemical Protocols, Humana Press, Totowa N.J.).

### Labeling of Molecules for Assay

A wide variety of labels and conjugation techniques are known by those skilled in the art and may be used in various nucleic acid, amino acid, and antibody assays. Synthesis of labeled molecules may be achieved using Promega (Madison Wis.) or Amersham Pharmacia Biotech kits for incorporation of a labeled nucleotide such as ³²P-dCTP, Cy3-dCTP or Cy5-dCTP or amino acid such as ³⁵S-methionine. Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

### Diagnostics

The nucleic acid molecules, fragments, oligonucleotides, complementary RNA and DNA molecules, and PNAs may be used to detect and quantify altered gene expression, absence/presence vs. excess, expression of mRNAs or to monitor mRNA levels during therapeutic intervention. Conditions, diseases or disorders associated with altered expression include epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas. The diagnostic assay may use hybridization or amplification technology to compare gene expression in a biological sample from a patient to standard samples in order to detect altered gene expression. Qualitative or quantitative methods for this comparison are well known in the art.

For example, the nucleic acid molecule or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

### Immunological Methods

Detection and quantification of a protein using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes is preferred, but a competitive binding assay may be employed (Coligan et al. (1997) Current Protocols in Immunology, Wiley-Interscience, New York N.Y.; Pound, supra).

### Therapeutics

Chemical and structural similarity, in the context of sequences and motifs, exists between regions of SEQ ID NOs:46 and 47 and other ASPs, human gastricsin (g1658286) and Neurospora crassa vacuolar protease A (g1039445). In addition, gene expression is closely associated with digestive system, reproductive and brain tissues, and in the pons from Alzheimer's disease and appears to play a role in conditions such as Alzheimer's disease and Down syndrome. In the treatment of conditions associated with increased expression or activity, it is desirable to decrease expression or protein activity. In the treatment of conditions associated with decreased expression or activity, it is desirable to increase expression or protein activity.

Chemical and structural similarity, in the context of sequences, exists between regions of variant nucleic acid molecules SEQ ID NOs: 1-42 and SEQ ID NOs:46 and 47. In addition, gene expression of the variant nucleic acid molecules is closely associated with digestive, reproductive, brain, and cardiovascular tissues, and appears to play a role in conditions such as Alzheimer's disease and Down syndrome.

In one embodiment, the mammalian protein or a portion or derivative thereof may be administered to a subject to treat or prevent a condition associated with altered expression or activity of the mammalian protein. Examples of such conditions include, but are not limited to, epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, an ectopic pregnancy, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas.

In another embodiment, a pharmaceutical composition comprising a substantially purified mammalian protein in conjunction with a pharmaceutical carrier may be administered to a subject to treat or prevent a condition associated with altered expression or activity of the endogenous protein including, but not limited to, those provided above.

In a further embodiment, a ligand which modulates the activity of the mammalian protein may be administered to a subject to treat or prevent a condition associated with altered lifespan, expression, or activity of the protein including, but not limited to, those listed above. In one aspect, an antibody which specifically binds the mammalian protein may be used as a targeting or delivery mechanism for bringing a pharmaceutical agent to cells or tissue that express the mammalian protein.

In an additional embodiment, a vector capable of expressing the mammalian protein or a portion or derivative thereof may be administered to a subject to treat or prevent a condition associated with altered lifespan, expression, or activity of protein including, but not limited to, those described above.

In a still further embodiment, a vector expressing the complement of the nucleic acid molecule or fragments thereof may be administered to a subject to treat or prevent a condition associated with altered lifespan, expression, or activity of the protein including, but not limited to, those described above.

Any of the nucleic acid molecules, complementary molecules and fragments thereof, proteins or portions thereof, vectors delivering these nucleic acid molecules or proteins, and their ligands may be administered in combination with other therapeutic agents. Selection of the agents for use in combination therapy may be made by one of ordinary skill in the art according to conventional pharmaceutical principles. A combination of therapeutic agents may act synergistically to affect prevention or treatment of a particular condition at a lower dosage of each agent.

### Modification of Gene Expression Using Nucleic Acids

Gene expression may be modified by designing complementary or antisense molecules (DNA, RNA, or PNA) to the control, 5′, 3′, or other regulatory regions of the mammalian gene. Oligonucleotides designed with reference to the transcription initiation site are preferred. Similarly, inhibition can be achieved using triple helix base-pairing that inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library of nucleic acid molecules or fragments thereof may be screened to identify those which specifically bind a regulatory, nontranslated sequence .

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features that would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, and or the modification of adenine, cytidine, guanine, thymine, and uridine with acetyl-, methyl-, thio- groups renders the molecule less available to endogenous endonucleases.

### Screening Assays

The nucleic acid molecule encoding the mammalian protein may be used to screen a library of molecules or compounds for specific binding affinity. The libraries may be DNA molecules, RNA molecules, PNAs, peptides, proteins such as transcription factors, enhancers, repressors, and other ligands that regulate the activity, replication, transcription, or translation of the nucleic acid molecule in the biological system. The assay involves combining the mammalian nucleic acid molecule or a fragment thereof with the library of molecules under conditions allowing specific binding, and detecting specific binding to identify at least one molecule that specifically binds the nucleic acid molecule.

Similarly the mammalian protein or a portion thereof may be used to screen libraries of molecules or compounds in any of a variety of screening assays. The portion of the protein employed in such screening may be free in solution, affixed to an abiotic or biotic substrate (e.g. borne on a cell surface), or located intracellularly. Specific binding between the protein and molecule may be measured. Depending on the kind of library being screened, the assay may be used to identify DNA, RNA, or PNA molecules, agonists, antagonists, antibodies, immunoglobulins, inhibitors, peptides, proteins, drugs, or any other ligand, which specifically binds the protein. One method for high throughput screening using very small assay volumes and very small amounts of test compound is described in U.S. Pat. No. 5,876,946, incorporated herein by reference, which screens large numbers of molecules for enzyme inhibition or receptor binding.

### Purification of Ligand

The nucleic acid molecule or a fragment thereof may be used to purify a ligand from a sample. A method for using a mammalian nucleic acid molecule or a fragment thereof to purify a ligand would involve combining the nucleic acid molecule or a fragment thereof with a sample under conditions to allow specific binding, detecting specific binding, recovering the bound protein, and using an appropriate agent to separate the nucleic acid molecule from the purified ligand.

Similarly, the protein or a portion thereof may be used to purify a ligand from a sample. A method for using a mammalian protein or a portion thereof to purify a ligand would involve combining the protein or a portion thereof with a sample under conditions to allow specific binding, detecting specific binding between the protein and ligand, recovering the bound protein, and using an appropriate chaotropic agent to separate the protein from the purified ligand.

### Pharmacology

Pharmaceutical compositions are those substances wherein the active ingredients are contained in an effective amount to achieve a desired and intended purpose. The determination of an effective dose is well within the capability of those skilled in the art. For any compound, the therapeutically effective dose may be estimated initially either in cell culture assays or in animal models. The animal model is also used to achieve a desirable concentration range and route of administration. Such information may then be used to determine useful doses and routes for administration in humans.

A therapeutically effective dose refers to that amount of protein or inhibitor that ameliorates the symptoms or condition. Therapeutic efficacy and toxicity of such agents may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it may be expressed as the ratio, LD50/ED50. Pharmaceutical compositions that exhibit large therapeutic indexes are preferred. The data obtained from cell culture assays and animal studies are used in formulating a range of dosage for human use.

### Model System

Animal models may be used as bioassays where they exhibit a toxic response similar to that of humans and where exposure conditions are relevant to human exposures. Mammals are the most common models, and most toxicity studies are performed on rodents such as rats or mice because of low cost, availability, and abundant reference toxicology. Inbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

### Toxicology

Toxicology is the study of the effects of agents on living systems. The majority of toxicity studies are performed on rats or mice to help predict the effects of these agents on human health. Observation of qualitative and quantitative changes in physiology, behavior, homeostatic processes, and lethality are used to generate a toxicity profile and to assess the consequences on human health following exposure to the agent.

Genetic toxicology identifies and analyzes the ability of an agent to produce genetic mutations. Genotoxic agents usually have common chemical or physical properties that facilitate interaction with nucleic acids and are most harmful when chromosomal aberrations are passed along to progeny. Toxicological studies may identify agents that increase the frequency of structural or functional abnormalities in progeny if administered to either parent before conception, to the mother during pregnancy, or to the developing organism. Mice and rats are most frequently used in these tests because of their short reproductive cycle which produces the number of organisms needed to satisfy statistical requirements.

Acute toxicity tests are based on a single administration of the agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Prolonged toxicity tests are based on the repeated administration of the agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

### Transgenic Animal Models

Transgenic rodents that over-express or under-express a gene of interest may be inbred and used to model human diseases or to test therapeutic or toxic agents. (See U.S. Pat. No. 4,736,866; U.S. Pat. No. 5,175,383; and U.S. Pat. No. 5,767,337). In some cases, the introduced gene may be activated at a specific time in a specific tissue type during fetal development or postnatally. Expression of the transgene is monitored by analysis of phenotype or tissue-specific mRNA expression in transgenic animals before, during, and after challenge with experimental drug therapies.

### Embryonic Stem Cells

Embryonic stem cells (ES) isolated from rodent embryos retain the potential to form an embryo. When ES cells are placed inside a carrier embryo, they resume normal development and contribute to all tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors for knockout strains contain a disease gene candidate modified to include a marker gene that disrupts transcription and/or translation in vivo. The vector is introduced into ES cells by transformation methods such as electroporation, liposome delivery, microinjection, and the like which are well known in the art. The endogenous rodent gene is replaced by the disrupted disease gene through homologous recombination and integration during cell division. Transformed ES cells are identified, and preferably microinjected into mouse cell blastocysts such as those from the C57BL/6 mouse strain. The blastocysts are surgically transferred to pseudopregnant dams and the resulting chimeric progeny are genotyped and bred to produce heterozygous or homozygous strains.

ES cells are also used to study the differentiation of various cell types and tissues in vitro, such as neural cells, hematopoietic lineages, and cardiomyocytes (Bain et al. (1995) Dev. Biol. 168:342-357; Wiles and Keller (1991) Development 111:259-267; and Kluget al. (1996) J. Clin. Invest. 98:216-224). Recent developments demonstrate that ES cells derived from human blastocysts may also be manipulated in vitro to differentiate into eight separate cell lineages, including endoderm, mesoderm, and ectodermal cell types (Thomson (1998) Science 282:1145-1147).

### Knockout Analysis

In gene knockout analysis, a region of a human disease gene candidate is enzymatically modified to include a non-mammalian gene such as the neomycin phosphotransferase gene (neo; Capecchi (1989) Science 244:1288-1292). The inserted coding sequence disrupts transcription and translation of the targeted gene and prevents biochemical synthesis of the disease candidate protein. The modified gene is transformed into cultured embryonic stem cells (described above), the transformed cells are injected into rodent blastulae, and the blastulae are implanted into pseudopregnant dams. Transgenic progeny are crossbred to obtain homozygous inbred lines.

### Knockin Analysis

Totipotent ES cells, present in the early stages of embryonic development, can be used to create knockin humanized animals (pigs) or transgenic animal models (mice or rats) of human diseases. With knockin technology, a region of a human gene is injected into animal ES cells, and the human sequence integrates into the animal cell genome by recombination. Totipotent ES cells that contain the integrated human gene are handled as described above. Inbred animals are studied and treated to obtain information on the analogous human condition. These methods have been used to model several human diseases. (See, e.g., Lee et al. (1998) Proc. Natl. Acad. Sci. 95:11371-11376; Baudoin et al. (1998) Genes Dev. 12:1202-1216; and Zhuang et al. (1998) Mol. Cell Biol. 18:3340-3349.)

### Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus monkeys (Macaca fascicularis, Macaca mulatta) and common marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPS are the first choice test animal. In addition, NHPS and individual humans exhibit differential sensitivities to many drugs and toxins and can be classified as “extensive metabolizers” and “poor metabolizers” of these agents.

In additional embodiments, the nucleic acid molecules that encode the mammalian protein may be used in any molecular biology techniques that have yet to be developed, provided the new techniques rely on properties of nucleic acid molecules that are currently known, including, but not limited to, such properties as the triplet genetic code and specific base pair interactions.

### EXAMPLES

It is to be understood that this invention is not limited to the particular machines, materials and methods described. Although particular embodiments are described, equivalent embodiments may be used to practice the invention. The described embodiments are not intended to limit the scope of the invention which is limited only by the appended claims. The examples below are provided to illustrate the subject invention and are not included for the purpose of limiting the invention. For purposes of example, the preparation of the human brain cDNA library, BRAINON01, is described.

I Representative cDNA Sequence Preparation

The BRAINON01 normalized cDNA library was constructed from cancerous brain tissue obtained from a 26-year-old Caucasian male (specimen #0003) during cerebral meningeal excision following diagnosis of grade 4 oligoastrocytoma localized in the right fronto-parietal part of the brain. Prior to surgery the patient was also diagnosed with hemiplegia, epilepsy, ptosis of eyelid, and common migraine. The frozen tissue was homogenized and lysed using a POLYTRON homogenizer (PT-3000; Brinkmann Instruments, Westbury N.Y.) in guanidinium isothiocyanate solution. The lysate was extracted with acid phenol, pH 4.7, per the RNA isolation protocol (Stratagene). The RNA was extracted with an equal volume of acid phenol, reprecipitated using 0.3 M sodium acetate and 2.5 volumes of ethanol, resuspended in DEPC-treated water, and treated with DNase for 25 min at 37° C. RNA extraction and precipitation were repeated as before. The mRNA was isolated with the OLIGOTEX kit (Qiagen, Chatsworth Calif.) and used to construct the cDNA library.

The mRNA was handled according to the recommended protocols in the SUPERSCRIPT plasmid system (Life Technologies). cDNA synthesis was initiated with a NotI-oligo d(T) primer. Double-stranded cDNA was blunted, ligated to SalI adaptors, digested with NotI, and fractionated on a SEPHAROSE CL4B column (Amersham Pharmacia Biotech), and those cDNAs exceeding 400 bp were ligated into the NotI and SalI sites of the PSPORT1 plasmid (Life Technologies). The plasmid was transformed into DH5α competent cells (Life Technologies).

II Normalization, Isolation, and Sequencing of cDNA Clones

4.9×10⁶ independent clones of the BRAINON01 plasmid library in E. coli strain DH12S (Life Technologies) were grown in liquid culture under carbenicillin (25 mg/l) and methicillin (1 mg/ml) selection following transformation by electroporation. To reduce the number of excess cDNA copies according to their abundance levels in the library, the cDNA library was then normalized in a single round according to the procedure of Soares et al. (1994, Proc. Natl. Acad. Sci. 91:9228-9232), with the following modifications. The primer to template ratio in the primer extension reaction was increased from 2:1 to 10:1. The ddNTP concentration in this reaction was reduced to 150 μM each ddNTP, allowing the generation of longer (400-1000 nt) primer extension products. The reannealing hybridization was extended from 13 to 48 hours. The single stranded DNA circles of the normalized library were purified by hydroxyapatite chromatography and converted to partially double-stranded by random priming, followed by electroporation into DH10B competent bacteria (Life Technologies).

Plasmid DNA was released from the cells and purified using the REAL Prep 96 plasmid kit (Qiagen). The recommended protocol was employed except for the following changes: 1) the bacteria were cultured in 1 ml of sterile Terrific Broth (Life Technologies) with carbenicillin at 25 mg/l and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were prepared using a MICROLAB 2200 (Hamilton, Reno Nev.) in combination with DNA ENGINE thermal cyclers (MJ Research, Watertown, Mass.). The cDNAs were sequenced by the method of Sanger and Coulson (1975, J Mol Biol 94:441-448) using ABI PRISM 377 DNA Sequencing systems (PE Biosystems), and reading frame was determined.

III Identification, Extension, Assembly, and Analyses

The consensus sequence (SEQ ID NO:43) was assembled from Incyte clones (library) 1863920X321F1 (PROSNOT19), 2233901H1 (PANCTUT02), 1611565T1 (COLNTUT06), 336541H1 (EOSIHET02), and the shotgun sequence, SBAA03902F1, from the LIFESEQ database (Incyte Pharmaceuticals) of human cDNA sequences and used to identify additional sequences in the LIFESEQ and ZOOSEQ databases (Incyte Pharmaceuticals) related to ASPs. Translation of SEQ ID NO:43 and 44 using MACDNASIS PRO software (Hitachi Software Engineering) elucidated the coding regions, SEQ ID NO:46 and 47. The nucleic acid and amino acid sequences were queried against databases such as the GenBank databases, SwissProt, BLOCKS, PRINTS, Prosite, and PFAM using BLAST. Motifs and HMM algorithms were used to perform functional analyses, and the antigenic index (Jameson-Wolf analysis) was determined using LASERGENE software (DNASTAR).

IV Sequence Similarity

Sequence similarity was calculated as percent identity based on comparisons between at least two nucleic acid molecules or amino acid sequences using the clustal method of the MEGALIGN program of LASERGENE software (DNASTAR). The clustal method uses an algorithm that groups sequences into clusters by examining the distances between all pairs. After the clusters are aligned pairwise, they are realigned in groups. Percent similarity between two sequences, sequence A and sequence B, is calculated by dividing the length of sequence A, minus the number of gap residues in sequence A, minus the number of gap residues in sequence B, into the sum of the residue matches between sequence A and sequence B, times one hundred. Gaps of very low or zero similarity between the two sequences are not included.

V Identification of Nucleic Acid Variants

Nucleic acid molecules which are splice variants of the nucleic acid molecules encoding the mammalian aspartyl protease (SEQ ID NOs:43 and 44) were identified by using BLAST or BLAST2 (Altschul, supra; NCI-BLASTN version 2.0.4 with default parameters), to identify clones in the LIFESEQ or ZOOSEQ database (Incyte Pharmaceuticals) which aligned with SEQ ID NOs:43 and 44. Mammalian nucleic acid molecule variants were selected by BLAST score. The BLAST score is calculated by scoring +5 for every base that matches in a nucleic acid High Scoring Pair (HSP) and −4 for every mismatch. The BLAST alignments were visually inspected and those clones with BLAST scores greater than 100 were aligned together using Phrap (Green, supra). Examples of nucleic acid sequences that flank splice junction sites of splice variants of nucleic acid sequences encoding proteins are well known in the art. Splice variant nucleic acid molecules were identified as those which aligned with 100% identity in a part of the nucleic acid sequence, and with less than 100% identity in the remaining part of the nucleic acid sequence and in which the initial nucleotide 3′ to the putative splice junction was a guanine base. A similar analysis of ESTs in the public domain identified clones that aligned with the nucleic acid sequences of human aspartyl protease and confirmed the location of the putative splice junctions.

VI Northern Analysis

Northern analysis is a laboratory technique used to detect the presence of a transcript of a gene and involves the hybridization of a labeled probe to a membrane on which RNAs from a particular cell type or tissue have been bound.

Analogous computer techniques applying BLAST2 (NCI-BLASTN version 2.0.4 with default parameters) were used to search for identical or related molecules in nucleotide databases such as the LIFESEQ database or ZOOSEQ database (both Incyte Pharmaceuticals). Sequence-based analysis is much faster than membrane-based hybridization, and the sensitivity of the computer search can be modified to determine whether any particular match is categorized as exact or similar. The basis of the BLAST search is the product score which is defined as: (percent sequence identity x percent maximum BLAST score) divided by 100. The product score takes into account both the degree of similarity between two sequences and the length of the sequence match. For example, with a product score of 40, the match will be exact within a 1% to 2% error, and with a product score of at least 70, the match will be exact. Similar or related molecules are usually identified by selecting those which show product scores between 8 and 40.

The results of northern analyses are reported as a fraction or a percentage distribution of libraries in which the transcript encoding the mammalian protein occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease categories included cancer, inflammation/trauma, cell proliferation, and neurological. For each category, the number of libraries expressing the sequence was counted and divided by the total number of libraries identified containing nucleic acid molecule variants of the nucleic acid molecule encoding the mammalian protein.

VII Extension of Nucleic Acid Molecules

At least one of the nucleic acid molecules used to assemble SEQ ID NOs:43 and 44 was produced by extension of an Incyte cDNA clone using oligonucleotide primers. One primer was synthesized to initiate 5′ extension of the known fragment, and the other, to initiate 3′ extension. The initial primers were designed using OLIGO 4.06 software (National Biosciences) to be about 22 to 30 nucleotides in length, to have a GC content of about 50%, and to anneal to the target sequence at temperatures of about 55° C. to about 68° C. Any fragment which would result in hairpin structures and primer-primer dimerizations was avoided. Selected human cDNA libraries were used to extend the molecule. If more than one extension is needed, additional or nested sets of primers are designed.

High fidelity amplification was obtained by performing PCR in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair selected from the plasmid: Step 1: 94° C., 3 min; Step 2: 94° C., 15 sec; Step 3: 60° C., 1 min; Step 4: 68° C., 2 min; Step 5: Steps 2, 3, and 4 repeat 20 times; Step 6: 68 ° C., 5 min; Step 7: storage at 4° C. In the alternative, parameters for the primer pair, T7 and SK+(Stratagene), were as follows: Step 1: 94° C., 3 min; Step 2: 94° C., 15 sec; Step 3: 57° C. 1 min; Step 4: 68° C., 2 min; Step 5: Steps 2, 3, and 4 repeated 20 times; Step 6: 68° C., 5 min; Step 7; storage at 4° C.

The concentration of DNA in each well was determined by dispensing 100 μl PICOGREEN quantitation reagent (0.25% (v/v); Molecular Probes) dissolved in 1×TE and 0.5 μl of undiluted PCR product into each well of an opaque fluorimeter plate (Corning Costar, Acton Mass.) and allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in producing longer sequence.

The extended sequences were desalted, concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested fragments were separated on about 0.6-0.8% agarose gels, fragments were excised as visualized under UV light, and agar removed/digested with AGARACE (Promega). Extended fragments were religated using T4 DNA ligase (New England Biolabs, Beverly Mass.) into pUC 18 vector (Amersham Pharmacia Biotech), treated with Pfu DNA polymerase (Stratagene) to fill-in restriction site overhangs, and transformed into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the conditions described above. Samples were diluted with 20% dimethysulphoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (PE Biosystems).

In like manner, a nucleic acid molecule of SEQ ID NOs: 1-44 is used to obtain regulatory sequences using the procedure above, oligonucleotides designed for outward extension, and a genomic DNA library.

VIII Labeling of Probes and Hybridization Analyses

Nucleic acids are isolated from a biological source and applied to a substrate for standard hybridization protocols by one of the following methods. A mixture of target nucleic acids, a restriction digest of genomic DNA, is fractionated by electrophoresis through an 0.7% agarose gel in 1×TAE [Tris-acetate-ethylenediamine tetraacetic acid (EDTA)] running buffer and transferred to a nylon membrane by capillary transfer using 20×saline sodium citrate (SSC). Alternatively, the target nucleic acids are individually ligated to a vector and inserted into bacterial host cells to form a library. Target nucleic acids are arranged on a substrate by one of the following methods. In the first method, bacterial cells containing individual clones are robotically picked and arranged on a nylon membrane. The membrane is placed on bacterial growth medium, LB agar containing carbenicillin, and incubated at 37° C. for 16 hours. Bacterial colonies are denatured, neutralized, and digested with proteinase K. Nylon membranes are exposed to UV irradiation in a STRATALINKER UV-crosslinker (Stratagene) to cross-ink DNA to the membrane.

In the second method, target nucleic acids are amplified from bacterial vectors by thirty cycles of PCR using primers complementary to vector sequences flanking the insert. Amplified target nucleic acids are purified using SEPHIACRYL-400 beads (Amersham Pharmacia Biotech). Purified target nucleic acids are robotically arrayed onto a glass microscope slide (Coming Science Products, Coming N.Y.). The slide is previously coated with 0.05% aminopropyl silane (Sigma-Aldrich) and cured at 110° C. The arrayed glass slide (microarray) is exposed to UV irradiation in a STRATALINKER UV-crosslinker (Stratagene).

cDNA probes are made from mRNA templates. Five micrograms of mRNA is mixed with 1 μg random primer (Life Technologies), incubated at 70° C. for 10 minutes, and lyophilized. The lyophilized sample is resuspended in 50 μl of 1×first strand buffer (cDNA Synthesis systems; Life Technologies) containing a dNTP mix, [α-³²P]dCTP, dithiothreitol, and MMLV reverse transcriptase (Stratagene), and incubated at 42° C. for 1-2 hours. After incubation, the probe is diluted with 42 μl dH₂O, heated to 95° C. for 3 minutes, and cooled on ice. mRNA in the probe is removed by alkaline degradation. The probe is neutralized, and degraded mRNA and unincorporated nucleotides are removed using a PROBEQUANT G-50 MicroColumn (Amersham Pharmacia Biotech). Probes can be labeled with fluorescent markers, Cy3-dCTP or Cy5-dCTP (Amersham Pharmacia Biotech), in place of the radionucleotide, [³²P]dCTP.

Hybridization is carried out at 65° C. in a hybridization buffer containing 0.5 M sodium phosphate (pH 7.2), 7% SDS, and 1 mM EDTA. After the substrate is incubated in hybridization buffer at 65° C. for at least 2 hours, the buffer is replaced with 10 ml of fresh buffer containing the probes. After incubation at 65° C. for 18 hours, the hybridization buffer is removed, and the substrate is washed sequentially under increasingly stringent conditions, up to 40 mM sodium phosphate, 1% SDS, 1 mM EDTA at 65° C. To detect signal produced by a radiolabeled probe hybridized on a membrane, the substrate is exposed to a PHOSPHORIMAGER cassette (Amersham Pharmacia Biotech), and the image is analyzed using IMAGEQUANT data analysis software (Amersham Pharmacia Biotech). To detect signals produced by a fluorescent probe hybridized on a microarray, the substrate is examined by confocal laser microscopy, and images are collected and analyzed using GEMTOOLS gene expression analysis software (Incyte Pharmaceuticals).

IX Complementary Nucleic Acid Molecules

Molecules complementary to the nucleic acid molecule, or a fragment thereof, are used to detect, decrease, or inhibit gene expression. Although use of oligonucleotides comprising from about 15 to about 30 base pairs is described, the same procedure is used with larger or smaller fragments or their derivatives (PNAs). Oligonucleotides are designed using OLIGO 4.06 software (National Biosciences) and SEQ ID NOs: 1-43. To inhibit transcription by preventing promoter binding, a complementary oligonucleotide is designed to bind to the most unique 5′ sequence, most preferably about 10 nucleotides before the initiation codon of the open reading frame. To inhibit translation, a complementary oligonucleotide is designed to prevent ribosomal binding to the mRNA encoding the mammalian protein.

X Expression of the Mammalian Protein

Expression and purification of the mammalian protein are achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, e.g., BL21(DE3). Antibiotic resistant bacteria express the mammalian protein upon induction with isopropyl beta-D-thiogalactopyranoside (IPTG). Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the mammalian cDNA by either homologous recombination or bacterial-mediated transposition involving transfer plasmid intermediates. Viral infectivity is maintained and the strong polyhedrin promoter drives high levels of cDNA transcription.

In most expression systems, the mammalian protein is synthesized as a fusion protein with, e.g., GST or a peptide epitope tag, such as FLAG, permitting rapid, single-step, affinity-based purification of recombinant fusion protein from crude cell lysates. GST enables the purification of fusion proteins on immobilized glutathione under conditions that maintain protein activity and antigenicity (Amersham Pharmacia Biotech). Following purification, the GST moiety can be proteolytically cleaved from the mammalian protein at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (Qiagen). Methods for protein expression and purification are discussed in Ausubel (supra, unit 16). Purified mammalian protein obtained by these methods can be used directly in the following activity assay.

XI Functional Assays

Serine protease activity of ASP is measured by the hydrolysis of various peptide thiobenzyl ester substrates. The substrates are chosen to represent the different SP types (chymase, trypase, aspase, etc.). Assays are performed at ambient temperature (˜25° C.) and contain an aliquot of ASP and the appropriate substrate in HEPES buffer, pH 7.5 containing 0.01M CaCl₂ and 8% dimethylsulfoxide. The reaction also contains 0.34 mM dithiopyridine which reacts with the thiobenzyl group that is released during hydrolysis and converts it to thiopyridone. The reaction is carried out in an optical cuvette, and the generation of thiopyridone is measured in a spectrophotometer by the absorption produced at 324 nm. The amount of thiopyridone produced in the reaction is proportional to the activity of ASP.

In the alternative, protein function is assessed by expressing the sequences encoding ASP at physiologically elevated levels in mammalian cell culture. The nucleic acid molecule is subcloned into PCMV SPORT vector (Life Technologies), which contains the strong cytomegalovirus promoter, and 5-10 μg of the vector is transformed into a endothelial or hematopoietic human cell line using transformation methods well known in the art. An additional 1-2 μg of a plasmid containing sequence encoding CD64-GFP (Clontech, Palo Alto Calif.) is co-transformed to provide a fluorescent marker to identify transformed cells using flow cytometry (FCM).

The influence of the introduced genes on expression can be assessed using purified populations of these transformed cells. Since CD64-GFP, which is expressed on the surface of transformed cells, binds to conserved regions of human immunoglobulin G (IgG), the transformed cells are separated using magnetic beads coated with either human IgG or antibody against CD64 (DYNAL, Lake Success N.Y.). mRNA is purified from the cells and analyzed by hybridization techniques.

XII Production of ASP Specific Antibodies

ASP is purified using polyacrylamide gel electrophoresis and used to immunize mice or rabbits. Antibodies are produced using the protocols below. Alternatively, the amino acid sequence of ASP is analyzed using LASERGENE software (DNASTAR) to determine regions of high immunogenicity. An immunogenic epitope, usually found near the C-terminus or in a hydrophilic region is selected, synthesized, and used to raise antibodies. Typically, epitopes of about 15 residues in length are produced using an ABI Model 431A peptide synthesizer (PE Biosystems) using Fmoc-chemistry and coupled to KLH (Sigma-Aldrich) by reaction with N-maleimidobenzoyl-N-hydroxysuccinimide ester to increase immunogenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occurring Protein Using Specific Antibodies

Naturally occurring or recombinant mammalian protein is substantially purified by immunoaffinity chromatography using antibodies specific for the protein. An immunoaffinity column is constructed by covalently coupling the antibody to CNBr-activated SEPHAROSE resin (Amersham Pharmacia Biotech). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the Nucleic Acid Molecule or Protein

The nucleic acid molecule, or fragments thereof, or the protein, or portions thereof, are labeled with ³²P-dCTP, Cy3-dCTP, or Cy5-dCTP (Amersham Pharmacia Biotech), or with BIODIPY or FITC (Molecular Probes, Eugene Oreg.), respectively. Libraries of candidate molecules previously arranged on a substrate are incubated in the presence of labeled nucleic acid molecule or protein. After incubation under conditions for either a nucleic acid or amino acid sequence, the substrate is washed, and any position on the substrate retaining label, which indicates specific binding or complex formation, is assayed, and the binding molecule is identified. Data obtained using different concentrations of the nucleic acid or protein are used to calculate affinity between the labeled nucleic acid or protein and the bound molecule.

XV Demonstration of Protein Activity

ASP, or biologically active fragments thereof, are labeled with ¹²⁵I Bolton-Hunter reagent (Bolton et al. (1973) Biochem. J. 133:529-539). Candidate ligand molecules previously arrayed in the wells of a multi-well plate are incubated with the labeled ASP, washed, and any wells with labeled ASP complex are assayed. Data obtained using different concentrations of ASP are used to calculate values for the number, affinity, and association of ASP with the candidate ligand molecules.

What is claimed is:

1. A substantially purified mammalian nucleic acid molecule and fragments thereof encoding ASPs, variants, and portions thereof, selected from:
a) an amino acid sequence of SEQ ID NO:46;
b) a naturally occurring amino acid sequence having a BLAST score of at least 250 bits when compared to the sequence of SEQ ID NO:46, wherein the BLAST score is calculated using NCI-BLASTX version 2.0.4;
c) a biologically active fragment of the amino acid sequence of SEQ ID NO:46; and
d) an immunologically active fragment of the amino acid sequence of SEQ ID NO:46.

2. An isolated and purified mammalian nucleic acid molecule or the complement thereof selected from:
a) a nucleic acid sequence of SEQ ID NOs:1-5, 7-29, 31-33, 37, and 43;
b) a naturally occurring nucleic acid sequence having a BLAST score of at least 200 bits when compared to a sequence of SEQ ID NOs:1-43, wherein the BLAST score is calculated using NCI-BLASTN version 2.0.4; and
c) a naturally occurring nucleic acid sequence having a BLAST score of between 200 and 400 bits when compared to a sequence of SEQ ID NO:44, wherein the BLAST score is calculated using NCI-BLASTN version 2.0.4.

3. A fragment of the mammalian nucleic acid sequence of claim 2(a).

4. A probe comprising at least 16 contiguous nucleic acids which hybridizes under high stringency conditions to the mammalian nucleic acid molecule of claim 2 or a complement or a fragment thereof.

5. A recombinant nucleic acid molecule comprising a promoter operably linked to the mammalian nucleic acid molecule of claim 2.

6. A cell transformed with the recombinant nucleic acid molecule of claim 5.

7. A method of producing a polypeptide, the method comprising:
a) culturing the cell of claim 6 under conditions for expression of the polypeptide, and
b) recovering the polypeptide so expressed.

8. A transgenic organism comprising the recombinant nucleic acid molecule of claim 5.

9. A method for detecting the mammalian nucleic acid molecule in a sample, the method comprising:
a) hybridizing the sample with the probe of claim 2 to form a hybridization complex; and
b) detecting the hybridization complex, wherein the hybridization complex indicates the presence of the mammalian nucleic acid molecule in the sample.

10. The method of claim 9 further comprising amplifying the nucleic acid molecule or a fragment thereof prior to hybridization.

11. A method of using the mammalian nucleic acid molecule or a fragment thereof to screen a library of molecules to identify at least one ligand that specifically binds the mammalian nucleic acid molecule, the method comprising:
a) combining the mammalian nucleic acid molecule of claim 2 with the library of molecules under conditions to allow specific binding, and
b) detecting specific binding, thereby identifying a ligand that specifically binds the mammalian nucleic acid molecule.

12. The method of claim 11 wherein the library is selected from DNA molecules, RNA molecules, PNAs, peptides, and proteins.

13. A method of using the mammalian nucleic acid molecule or a fragment thereof to purify a ligand that specifically binds the mammalian nucleic acid molecule from a sample, the method comprising:
a) combining the mammalian nucleic acid molecule or a fragment thereof of claim 2 with a sample under conditions to allow specific binding;
b) recovering the bound mammalian nucleic acid molecule; and
c) separating the mammalian nucleic acid molecule from the ligand, thereby obtaining purified ligand.

14. A substantially purified protein or a portion thereof selected from:
a) an amino acid sequence of SEQ ID NO:46;
b) a naturally occurring amino acid sequence having a BLAST score of at least 250 bits when compared to a sequence of SEQ ID NO:46, wherein the BLAST score is calculated using NCI-BLASTX version 2.0.4;
c) a biologically active fragment of the amino acid sequence of SEQ ID NO:46; and
d) an immunologically active fragment of the amino acid sequence of SEQ ID NO:46.

15. A method for using a protein or a portion thereof to screen a library of molecules to identify at least one ligand that specifically binds the protein, the method comprising:
a) combining the protein or a portion thereof of claim 14 with the library of molecules under conditions to allow specific binding, and
b) detecting specific binding, thereby identifying a ligand that specifically binds the protein.

16. The method of claim 15 wherein the library is selected from DNA molecules, RNA molecules, PNAs, peptides, proteins, agonists, antagonists, antibodies, immunoglobulins, inhibitors, drug compounds and pharmaceutical agents.

17. A method of using a protein or a portion thereof to purify a ligand that specifically binds the protein from a sample, the method comprising:
a) combining the protein or a portion thereof of claim 14 with a sample under conditions to allow specific binding,
b) recovering the bound protein, and
c) separating the protein from the ligand, thereby obtaining purified ligand.

18. A pharmaceutical composition comprising an effective amount of the protein of claim 14 and a pharmaceutically acceptable excipient.

19. A method for treating a disease or condition associated with altered expression of ASP, comprising administering to a patient in need of such treatment the pharmaceutical composition of claim 18.

20. The method of claim 19, wherein the disease or condition is selected from Alzheimer's disease and Down syndrome.
